(19) United States
(12) Patent Application Publication
(10) Pub. No.: US 2023/0331841 A1
(43) Pub. Date: Oct. 19, 2023

(54) ANTI-CD83 CAR-T CELLS WITH REGNASE-1 AND/OR TGFBRII DISRUPTION

(71) Applicant: CRISPR Therapeutics AG, Zug (CH)

(72) Inventors: Daniel Robert HOSTETTER, Cambridge, MA (US); Shweta SINGH, Cambridge, MA (US); Jonathan TERRETT, Cambridge, MA (US)

(21) Appl. No.: 18/188,406

(22) Filed: Mar. 22, 2023

Related U.S. Application Data
(60) Provisional application No. 63/322,868, filed on Mar. 23, 2022.
(60) Provisional application No. 63/417,775, filed on Oct. 20, 2022.

(51) Int. Cl. C07K 16/28; C12N 5/0783; C12N 9/02; C07K 14/71; C12N 9/22; C12N 15/11; C07K 14/74; C07K 14/705; A61K 38/17; A61K 39/395; A61K 39/00; A61P 35/02

(52) U.S. Cl. CPC C07K 16/2803; C12N 5/0636; C12N 9/0071; C07K 14/71; C12N 9/22; C12N 15/11; C07K 14/70539; C07K 14/70578; C07K 16/2818; C07K 16/2809; A61K 38/1774; A61K 38/177; A61K 39/3955; A61K 39/4611; A61K 39/4631; A61K 39/46433; A61K 39/464411; A61P 35/02; C07K 2317/622; C07K 2317/565; A61K 2039/505; C12N 2510/00; C07K 2319/02; C12N 2800/80; C12N 2310/20; A61K 2039/5156; A61K 2239/22; A61K 2239/13; C07K 2319/30

(57) ABSTRACT

A population of genetically engineered T cells, comprising a nucleic acid encoding an anti-CD83 CAR, a disrupted Reg1 gene, and/or a disrupted TGFBRII gene. Such genetically engineered T cells may comprise further genetic modifications, for example, a disrupted CD83 gene. The population of genetically engineered T cells exhibit one or more of (a) improved cell growth activity; (b) enhanced persistence; and (c) reduced T cell exhaustion, (d) enhanced cytotoxicity activity, (e) resistant to inhibitory effects induced by TGF-β, and (f) resistant to inhibitory effects by fibroblasts and/or inhibitory factors secreted thereby, as compared to non-engineered T cell counterparts.

### CROSS REFERENCE TO RELATED APPLICATIONS

This application claims the benefit of the filing dates of U.S. Provisional Application No. 63/322,868, filed Mar. 23, 2022, and U.S. Provisional Application No. 63/417,775, filed Oct. 20, 2022, the entire contents of each of which are incorporated by reference herein.

### SEQUENCE LISTING

The instant application contains a Sequence Listing which has been filed electronically in XML format and is hereby incorporated by reference in its entirety. Said XML copy, created on Mar. 15, 2023, is named 095136-0741-063WO1_SEQ.XML and is 111,265 bytes in size.

### BACKGROUND OF THE INVENTION

Chimeric antigen receptor (CAR) T-cell therapy uses genetically modified T cells to more specifically and efficiently target and kill pathogenic cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

T cells having improved persistence in culture are desired in CAR T therapy. Such T cells live longer in both in vitro and in vivo, thereby conferring benefits in CAR T cell manufacturing and clinical applications. However, it remains challenging to improve persistence of T cells in culture.

### SUMMARY OF THE INVENTION

The present disclosure is based, at least in part, on the development of genetically edited anti-CD83 CAR-T cells carrying a disrupted Regnase 1 (Reg1) gene (e.g., “Reg1 Knockout T cells”), a disrupted TGFBRII gene (e.g., “TGFBRII Knockout T cells”), and optionally a disrupted CD83 gene (e.g., “CD83 Knockout T cells”), a disrupted TRAC gene (e.g., “TRAC Knockout T cells”), a disrupted β2M gene (e.g., “β2M knockout T cells”), or a combination thereof. It was reported herein that disruption of the Reg1 gene and TGFBRII gene enhanced the cytotoxicity of the anti-CD83 CAR-T cells both in vitro and in vivo without affecting features of the CAR-T cells such as cell growth, CD4:CD8 ratio, and/or expression levels of immune checkpoint markers. Further, the genetically edited anti-CD83 CAR-T cells as disclosed herein showed enhanced T-cell expansion and improved anti-tumor activity.

Accordingly, the present disclosure provides, in some aspects, a population of genetically engineered T cells, comprising: (i) a nucleic acid encoding a chimeric antigen receptor (CAR) that binds CD83 (anti-CD83 CAR); and (ii) a disrupted Regnase-1 (Reg1) gene and/or a disrupted Transforming Growth Factor Beta Receptor II (TGFBRII) gene. In some embodiments, the population of genetically engineered T cells comprises both the disrupted Reg1 gene and the disrupted TGFBRII gene.

Any of the genetically engineered T cells may further comprise (iii) a disrupted CD83 gene. Alternatively or in addition, the population of genetically engineered T cells may further comprise (iv) a disrupted T cell receptor alpha chain constant region (TRAC) gene, a disrupted beta-2-microglobulin (β2M) gene, or a combination thereof.

In some embodiments, the population of genetically engineered T cells may comprise (i) the nucleic acid encoding the anti-CD83 CAR; (ii) the disrupted Reg1 gene and the disrupted TGFBRII gene; (iii) the disrupted CD83 gene; and (iv) the disrupted TRAC gene and the disrupted β2M gene.

In some embodiments, the disrupted Reg1 gene is genetically edited in exon 4. Alternatively, or in addition, the disrupted TGFBRII gene is genetically edited in exon 5. Still alternatively, or in addition, the disrupted CD83 gene is genetically edited in exon 2.

In some embodiments, the disrupted CD83 gene, the disrupted Reg1 gene, the disrupted TGFBRII gene, the disrupted TRAC gene, and/or the disrupted β2M gene can be genetically edited by a CRISPR/Cas-mediated gene editing system.

In some instances, the CRISPR/Cas-mediated gene editing comprises a guide RNA (gRNA) targeting a site in the Reg1 gene that comprises a nucleotide sequence of SEQ ID NO: 36 or 37. In some examples, the gRNA targeting the Reg1 gene comprises a spacer, which comprises the nucleotide sequence of SEQ ID NO: 34.

In some instances, the CRISPR/Cas-mediated gene editing system comprises a guide RNA (gRNA) targeting a site in the TGFBRII gene that comprises a nucleotide sequence of SEQ ID NO: 30 or 31. In some examples, the gRNA targeting the TGFBRII gene comprises a spacer, which comprises the nucleotide sequence of SEQ ID NO:28.

In some instances, the CRISPR/Cas-mediated gene editing system comprises a guide RNA (gRNA) targeting a site in the CD83 gene that comprises a nucleotide sequence of SEQ ID NO: 24 or 25. In some examples, the gRNA targeting the CD83 gene comprises a spacer, which comprises the nucleotide sequence of SEQ ID NO: 22. Alternatively, the CRISPR/Cas-mediated gene editing system comprises a guide RNA (gRNA) targeting a site in the CD83 gene that comprises a nucleotide sequence of SEQ ID NO: 100 or 101. In some examples, the gRNA targeting the CD83 gene comprises a spacer, which comprises the nucleotide sequence of SEQ ID NO: 98.

Any of the populations of genetically engineered T cells disclosed herein may further comprise a disrupted T cell receptor alpha chain constant region (TRAC) gene, and/or a disrupted beta-2-microglobulin (β2M) gene. In some embodiments, the T cells comprise a disrupted T cell receptor alpha chain constant region (TRAC) gene. Alternatively, or in addition, the T cells comprise a disrupted beta-2-microglobulin (β2M) gene. In some examples, the disrupted TRAC gene, and/or the disrupted β2M gene is genetically edited by one or more CRISPR/Cas-mediated gene editing system.

In some instances, the CRISPR/Cas-mediated gene editing comprises a guide RNA (gRNA) targetinga site in the TRAC gene that comprises a nucleotide sequence of SEQ ID NO: 6 or 7. In some examples, the gRNA targeting the TRAC gene comprises a spacer, which comprises the nucleotide sequence of SEQ ID NO: 4.

In some instances, the CRISPR/Cas-mediated gene editing system comprises a guide RNA (gRNA) targeting a site in the β2M gene that comprises a nucleotide sequence of SEQ ID NO: 12 or 13. For example, the gRNA may comprise a spacer sequence comprising the nucleotide sequence of SEQ ID NO:10. Alternatively, the CRISPR/Cas-mediated gene editing system comprises a guide RNA (gRNA) targeting a site in the β2M gene that comprises a nucleotide sequence of SEQ ID NO: 18 or 19. For example, the gRNA may comprise a spacer sequence comprising the nucleotide sequence of SEQ ID NO:16.

In some embodiments, the nucleic acid encoding the anti-CD83 CAR is inserted in the genome of the T cells. In some instances, the nucleic acid encoding the CAR is inserted in the disrupted CD83 gene, the disrupted Reg1 gene, the disrupted TGFBRII gene, the disrupted TRAC gene, or the disrupted β2M. In some examples, the nucleic acid encoding the CAR is inserted in the disrupted TRAC gene. In specific examples, the nucleic acid encoding the CAR may replace the deleted fragment comprising SEQ ID NO:7 in the TRAC gene.

Any of the CAR constructs disclosed herein may comprise an extracellular antigen binding domain specific to CD83, a co-stimulatory signaling domain of 4-1BB or CD28, and a cytoplasmic signaling domain of CD3ζ. In some embodiments, the extracellular antigen binding domain is a single chain variable fragment (scFv) that binds CD83 (anti-CD83 scFv). Such ananti-CD83 scFv may comprise a heavy chain variable region (V_{H}) and a light chain variable region (V_{L}). In some instances, the V_{H} comprises heavy chain complementary determining region (CDR) 1, CDR2, and CDR3 set forth as SEQ ID NOs:71, 72, and 73, respectively. Alternatively, or in addition, the V_{L} comprises light chain complementary determining region (CDR) 1, CDR2, and CDR3 set forth as SEQ ID NOs:74, 75 and 76, respectively. In some instances, the V_{H} comprises the amino acid sequence of SEQ ID NO:77, and/or the V_{L} comprises the amino acid sequence of SEQ ID NO:78. In some instances, the anti-CD83 scFv comprises the amino acid sequence of SEQ ID NO:79. In some instances, the CAR that binds CD83 comprises the amino acid sequence of SEQ ID NO:80 or 81.

The genetically engineered T cells disclosed herein may be derived from primary T cells of one or more human donors. In some instances, the genetically engineered T cells show cytokine-dependent growth.

In some embodiments, the genetically engineered T cells disclosed herein may further express a chimeric antigen receptor (CAR) that binds a tumor antigen, which optionally is CD19, BCMA, or CD70.

In other aspects, the present disclosure provides a method for preparing any of the populations of genetically engineered T cells disclosed herein. In some instances, the method may comprise: (a) providing a plurality of cells, which are T cells or precursor cells thereof; (b) delivering to the T cells a nucleic acid encoding a chimeric antigen receptor (CAR) that binds CD83 (“anti-CD83 CAR”), e.g., those disclosed herein; (c) genetically editing a Reg1 gene, a TGFBRII gene, or a combinatuion thereof, thereby producing a population of genetically engineered T cells expressing the anti-CD83 CAR and having a Reg1 gene, a disrupted, TGFBRII gene or a combination thereof.

In some embodiments, the plurality of cells in (a) comprises a disrupted CD83 gene, a disrupted TRAC gene, a disrupted β2M gene, or a combination thereof. In some embodiments, the method further comprises: (d) genetically editing a CD83 gene, and/or (e) genetically editing a TRAC gene, a β2M gene, or a combination thereof.

Any of steps (c), (d), and/or (e) can be performed by one or more CRISPR/Cas-mediated gene editing systems. In some embodiments, an RNA-guided nuclease and one or more gRNAs targeting the Reg1 gene, the TGFBRII gene, the CD83 gene, the TRAC gene, and/or the β2M gene are delivered to the plurality of cells. In some embodiments, the RNA-guided nuclease and the one or more gRNAs form one or more ribonucleoprotein particles (RNPs). In some instances, the RNA-guided nuclease is a CRISPR/Cas9 nuclease. In some specific instances, the Cas9 nuclease is a S. pyogenes Cas9 nuclease.

In some embodiments, the nucleic acid encoding the anti-CD83 CAR is in an AAV vector. In some embodiments, the nucleic acid encoding the anti-CD83 CAR comprises a left homology arm and a right homology arm flanking the nucleotide sequence encoding the CAR, and the left homology arm and the right homology arm are homologous to a genomic locus in the T cells, which allows for insertion of the nucleic acid into the genomic locus. In some instances, the genomic locus is in the CD83 gene, in the Reg1 gene, in the TGFBRII gene, in the TRAC gene, or in the β2M gene. In some specific instances, the genomic locus is in the TRAC gene.

In some embodiments, the genetic editing is performed by delivering (a) a guide RNA (gRNA) targeting a site in the Reg1 gene, e.g., the site that comprises the nucleotide sequence of SEQ ID NO: 37, (b) a gRNA targeting a site in the TGFBRII gene, e.g., the site that comprises the nucleotide sequence of SEQ ID NO: 31, or a combination of (a) and (b) to the plurality of T cells. In some instances, the genetic editing is further performed by delivering a gRNA targeting a site in the CD83 gene, e.g., the site that comprises the nucleotide sequence of SEQ ID NO:25 or the site that comprises the nucleotide sequence of SEQ ID NO: 101 to the plurality of T cells. In some instances, the genetic editing may be further performed by delivering a gRNA targeting a site in the TRAC gene, e.g., the site that comprises the nucleotide sequence of SEQ ID NO:7, and/or a gRNA targeting a site in the β2M gene, e.g., the site that comprises the nucleotide sequence of SEQ ID NO: 13 or 19, to the plurality of T cells. Any of the gRNAs targeting the Reg1 gene, the TGFBRII gene, the CD83 gene, the TRAC gene, and/or the β2M gene as disclosed herein can be used in the methods disclosed herein for producing the genetically edited anti-CD83 CAR-T cells.

Any population of the genetically engineered T cells prepared by a method disclosed herein is also within the scope of the present disclosure.

Further, the present disclosure provides a method for eliminating undesired cells in a subject, the method comprising administering to a subject in need thereof any of the populations of genetically engineered T cells disclosed herein. In some embodiments, the undesired cells are disease cells expressing CD83. In some instances, the disease cells expressing CD83 are CD83+ cancer cells or CD83+ autoreactive immune cells.

In some embodiments, the subject is a human patient having a cancer or an autoimmune disorder. In some embodiments, the population of genetically engineered T cells is allogeneic to the subject.

Also within the scope of the present disclosure are any of the genetically engineered T cells, gRNAs targeting CD83, gRNAs targeting Reg1, and/or gRNAs targeting TGFBRII for use in treating a target disease as disclosed herein, or uses of such for manufacturing a medicament for the intended therapeutic purposes.

Further within the scope of the prsent disclosure is a method of suppressing immune responses in a subject, the method comprising: administering to a subject in need thereof (a) an effective amount of a population of genetically engineered T cells as described above and herein, and (b) an effective amount of an anti-inflammatory agent.

In some embodiments, the population of genetically engineered T cells and the anti-inflammatory agent (e.g., an anti-CTLA-4-Fc fusion polypeptide such as belatacept) are formulated in two separate compositions. In some examples, the two compositions are administered to the subject in need of the treatment concurrently. Alternatively, the two compositions are administered to the subject sequentially.

In some embodiments, the anti-inflammatory agent is an antibody that inhibits a pro-inflammatory cytokine. Alternatively, the anti-inflammatory agent may be an agent that inhibits T-cell co-stimulation. In some embodiments, the anti-inflammatory agent is a CTLA4-Fc fusion protein. In some examples, the CTLA4-Fc fusion protein is belatacept. Alternatively, the CTLA-4-Fc fusion protein is abatacept.

In some embodiments, the subject is a human patient. Such a human patient may have or at risk for an immune disease, e.g., an autoimmune disease, or a graft-versus-host disease (GvHD). In some examples, the human patient has an autoimmune disease, e.g., those disclosed herein. In other examples, the human patient has or is at risk for GvHD.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 illustrates assessment of CD83 CAR T-cell expansion with or without genetic disruption of the Reg1 and TGFBRII genes. No RNP are control cells. CD83 CAR: 83 KO are CD83 knock-out cells that express the anti-CD83 CAR. CD83 CAR: 83+R+T KO are CD83+Reg1+TGFBRII knock-out cells that express the anti-CD83 CAR.

FIGS. 2A-2B illustrate the frequency of CD4 and CD8 T cells (CD4/CD8) in CD83 CAR T-cell cohorts determined by flow cytometry on Day 6 (FIG. 2A) and Day 13 (FIG. 2B).

FIGS. 3A-3B illustrate in vitro cytotoxicity of anti-CD83 CAR T cells. FIG. 3A: the number of viable target cells counted on Day 2, Day 5, Day 7 and Day 10. FIG. 3B: the number of T cells counted on Day 2, Day 5, Day 7 and Day 10.

FIGS. 4A-4C illustrate in vivo cytotoxicity of anti-CD83 CAR T cells. FIG. 4A: tumor growth in mice with no treatment. FIG. 4B: tumor growth in mice treated with CD83 CAR: 83 KO T cells. FIG. 4C: tumor growth in mice treated with CD83 CAR: 83+R+T KO T cells.

FIGS. 5A-5C illustrate enhancement of the anti-tumor activity delivered by anti-CD83 CAR-T cells with the CD83 knockout. FIG. 5A: tumor growth in mice treated with CD83 CAR: 83 KO T cells. FIG. 5B: tumor growth in mice treated with CD83 CAR: 83+R+T KO T cells. FIG. 5C: probability of survival of mice unretreated or treated with anti-CD83 CAR T cells.

FIGS. 6A-6C illustrate results of a tumor rechallenge after CAR T-cell treatment. FIG. 6A: tumor growth in mice rechallenged with THP-1 tumor cells 45 days after CD83 CAR: 83 KO T cell treatment compared to no treatment group. FIG. 6B: tumor growth in mice rechallenged with THP-1 tumor cells 45 days after CD83 CAR: 83 KO+R/T KO T cell treatment compared to no treatment group. FIG. 6C: probability of survival of mice rechallenged with THP-1 tumor cells 45 days after CAR-T treatment compared to no treatment group.

FIG. 7 is a diagram shown that anti-CD83 CART cells with disruption of the CD83, Regnase-1, and TGFBRII genes improved animal survival rates in a mouse graft-versus-host disease (GvHD) model, as compared with the counterpart anti-CD83 CAR-T cells with no CD83 gene disruption or with CD83 gene disruption alone.

FIG. 8 is a diagram shown that anti-CD83 CAR T cells with CD83, Regnase-1, and TGFBRII gene disruption enhanced survival rates in a mouse THP1 xenograft tumor model, as compared with the counterpart anti-CD83 CAR-T cells with no CD83 gene disruption or with CD83 gene discruption alone.

FIGS. 9A-9D include diagrams showing body weight changes over the course of treatment in mice of various treatment groups. FIG. 9A: untreated animals. FIG. 9B: animals injected with CD83 CAR-T KO cells alone. FIG. 9C: animals injected with belatacept alone. FIG. 9D: animals injected with CD83 CAR-T KO cells and belatacept in combination.

FIG. 10 is a diagram showing survival curves for varioustreatment groups as indicated.

FIGS. 11A-11B include diagrams showing representative engraftment analysis for the treatment groups as indicated. FIG. 11A: levels of CD45+ cells in animals injected with CD83 CAR-T KO cells with or without belatacept. FIG. 11B: levels of CD3+ cells in animals injected with CD83 CAR-T KO and belatacept.

### DETAILED DESCRIPTION OF THE INVENTION

The present disclosure aims at establishing genetically engineered anti-CD83 CAR-T cells having improved growth activity, persistence, and enhanced cytotoxicity against target cells (for example, CD83+ tumor cells such as myeloid leukemia cells). It is also reported herein that the combination therapy of anti-CD83 CAR-T cells and belatacept (as an example of anti-inflammatory agents) sucussfully inhibited or delayed development of graft-versus-host disease (GvHD) and prolonged survival rates as observed in an animal model. Accordingly, genetically engineered anti-CD83 CAR-T cells in combination with an anti-inflammatory agent as disclosed herein are expected to be more effective in suppressing immune responses, thereby benefiting treatment of immune disorders associated with undesired immune responses, such as GvHD and autoimmune diseases.

Genetically engineered T cells disclosed herein, having a nucleic acid encoding a chimeric antigen receptor (CAR) that binds CD83 (anti-CD83 CAR), a disrupted TGFBRII gene, a disrupted Reg1 gene, or a combination thereof, and optionally one or more additional genetic edits, for example, a disrupted CD83 gene, a disrupted TRAC gene, and/or a disrupted β2M gene, as provided herein showed improved growth/expansion capacity upon activation and enhanced cytotoxicity both in vitro and in vivo relative to counterpart CAR-T cells having wild-type Reg1 and TGFBRII genes. Further, genetic editing of the Reg1 and TGFBRII genes were found to have no negative impact on the resultant CAR-T cells, including CD4/CD8 ratio and levels of immune checkpoint markers. See also PCT-Patent Application No. WO2022064428, the relevant disclosures thereof are incorporated by reference for the subject matter and purpose referenced herein. Disruption of CD83 gene would improve growth of anti-CD83 CAR-T cells, reduce production of pro-inflammatory cytokines, and/or enhance potency of the resultant anti-CD83 CAR-T cells. See, also International Application No. PCT/IB2022/058633, filed on September 13, 2022, the relevant disclosures thereof are incorporated by references for the subject matter and purpose referenced herein.

Accordingly, provided herein are genetically engineered anti-CD83 T cells having disrupted Reg1 gene and/or TGFBRII gene, and optionally disrupted CD83 gene, methods of producing such T cells, and methods of using such T cells for therapeutic uses. In some instances, the anti-CD83 CAR-T cells may be allogeneic CAR-T cells having further genetic edits, for example, a disrupted TRAC gene, a disrupted β2M gene, or a combination thereof. The anti-CD83 CAR-T cells disclosed herein may confer one or more benefits in both CAR-T cell manufacturing (e.g., enhanced capacity in cell growth and expansion in vitro) and clinical applications (e.g., enhanced therapeutic efficacy, enhanced in vivo expansion and persistence capacitiy, and/or reduced risk of graft-versus-host disease). Components and processes (e.g., the CRISPR approach for gene editing and components used therein) for making the T cells disclosed herein are also within the scope of the present disclosure.

### I. Genetically Engineered T Cells Having Enhanced Features

The T cells disclosed herein comprises genetically engineered T cells having enhanced persistence in culture. Such genetically engineered T cells may have genetic editing of the Reg1 gene, and/or the TGFBRII gene, and optionally the CD83 gene. In some instances, such genetically engineered T cells may have genetic editing of both the Reg1 gene and the TGFBRII gene. In some instances, such genetically engineered T cells may have genetic editing of the CD83 gene, the Reg1 gene and the TGFBRII gene.

In some examples, the genetically engineered T cells may have further genetic edits for producing allogeneic T cells, e.g., genes involved in graft-versus-host or host-versus-grant immune responses. For example, the genetically engineered T cells may have disrupted TRAC gene, disrupted β2M gene, or a combination thereof. In specific examples, the genetically engineered T cells have disrupted TRAC and β2M genes.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

In some embodiments, the genetically engineered T cells carry a disrupted Reg1 gene, and/or a disrupted TGFBRII gene, and optionally a disrupted CD83 gene. Such genetically engineered T cells may further comprise one or more disrupted genes, for example, TRAC, β2M, or a combination thereof. Such genetically engineered T cells may further express a chimeric antigen receptor (CAR), which may be capable of binding to an antigen of interest, for example, a tumor associated antigen (e.g., CD83). In some instances, the genetically engineered T cells carry the nucleic acid encoding the anti-CD83 CAR; the disrupted Reg1 gene and the disrupted TGFBRII gene; the disrupted CD83 gene, the disrupted TRAC gene and the disrupted β2M gene. Surprisingly, it was reported herein that introducing the multiple genetic edits into T cells showed no negative impact on CAR-T cell features and resulted in improved therapeutic potency and enhanced cell growth/expansion upon activation.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

(a) Genetically Edited Genes

In some aspects, the present disclosure provides genetically engineered T cells that may comprise a nucleic acid encoding a chimeric antigen receptor (CAR) that binds CD83 (anti-CD83 CAR), and a disrupted Reg1 gene, a disrupted TGFBRII gene, or a combination thereof. In some embodiments, the genetically engineered T cells provided herein comprise both a disrupted Reg1 gene and a disrupted TGFBRII gene. In some instances, the genetically engineered T cells disclosed herein may further comprise a disrupted CD83 gene, a disrupted β2M gene, a disrupted TRAC gene, or a combination thereof.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

Reg1 Gene Editing

In some embodiments, the genetically engineered T cells may comprise a disrupted gene involved in mRNA decay. Such a gene may be Reg1. Reg1 contains a zinc finger motif, binds RNA and exhibits ribonuclease activity. Reg1 plays roles in both immune and non-immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some instances, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some instances, one or more genetic editing may occur in exon 4. Such genetic editing may be induced by the CRISPR/Cas technology using a suitable guide RNA, for example, a gRNA targeting the ACGACGCGTGGGTGGCAAGC (SEQ ID NO:37) site in the Reg1 gene. Exemplary gRNAs targeting the Reg1 gene are provided in Table 17 below. In some embodiments, an edited Reg1 gene may comprise one or more nucleotide sequences selected from those listed in Table 21, which may be generated using a single gRNA (e.g., those listed in Table 17). See also WO2022064428, the relevant disclosures are incorporated by reference herein for the subject matter and purpose referenced herein.

Disruption of the Reg1 gene can enhance long-term-persistence and maintain robust effector function, thereby improving T cell functionality.

TGFBRII Gene Editing

In some embodiments, the genetically engineered T cells may comprise a disrupted TGFBRII gene, which encodes Transforming Growth Factor Receptor Type II (TGFBRII). TGFBRII receptors are a family of serine/threonine kinase receptors involved in the TGFb signaling pathway. These receptors bind growth factor and cytokine signaling proteins in the TGFb family, for example, TGFs (TGFβ1, TGFβ2, and TGFβ3), bone morphogenetic proteins (BMPs), growth differentiation factors (GDFs), activin and inhibin, myostatin, anti-Mullerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some instances, one or more genetic editing may occur in exon 5. Such genetic editing may be induced by a gene editing technology, (e.g., the CRISPR/Cas technology) using a suitable guide RNA, for example, a gRNA targeting the CCCCTACCATGACTTTATTC (SEQ ID NO:31) site in the TGFBRII gene. Exemplary gRNAs targeting the Reg1 gene are provided in Table 17 below. In some embodiments, an edited TGFBRII gene may comprise one or more nucleotide sequences selected from those listed in Table20, which may be generated using a single gRNA (e.g., those listed in Table 17). See also WO2022064428, the relevant disclosures are incorporated by reference herein for the subject matter and purpose referenced herein.

Disruption of the TGFBRII gene can eliminate surface expression of TGFBRII and reduce the immunosuppressive effect of transforming growth factor beta (TFG-β) in the tumor microenvironment.

CD83 Gene Editing

In some embodiments, the genetically engineered T cells may comprise a disrupted CD83 gene. CD83 is a member of the immunoglobulin (Ig) superfamily and is expressed in membrane bound or soluble forms. The membrane-bound CD83 contains an extracellular V-type immunoglobulin-like domain, a transmembrane domain and a cytoplasmic signaling domain. The soluble form contains only the -type immunoglobulin-like domain. The gene encoding CD83 is located on human chromosome 6p23. The structure of the human CD83 gene is known in the art, e.g., under Gene ID ENSG00000112149.

CD83 is expressed in various types of immune cells, including regulatory T cells, dendritic cells, B cells, and T cells. It was reported that CD83 may involve in inflammation and serves as a binding site for the aryl hydrocarbon receptor.

In some examples, the genetically engineered T cells may comprise a disrupted CD83 gene such that the expression of CD83 in the T cells is substantially reduced or eliminated completely. The disrupted CD83 gene may comprise one or more genetic edits at one or more suitable target sites (e.g., in coding regions or in non-coding regulatory regions such as promoter regions) that disrupt expression of the CD83 gene. Such target sites may be identified based on the gene editing approach for use in making the genetically engineered T cells. Exemplary target sites for the genetic edits may include exon 2, exon 3, or a combination thereof. In some examples, one or more genetic editing may occur in exon 2. Such genetic editing may be induced by a gene editing technology, (e.g., the CRISPR/Cas technology) using a suitable guide RNA, for example, a gRNA targeting the GTAGGGAACCTGCGGATCCCAGG (SEQ ID NO:25) site in the CD83 gene. Exemplary gRNAs targeting the Reg1 gene are provided in Table 17 below. See, also International Application No. PCT/IB2022/058633, filed on September 13, 2022, the relevant disclosures thereof are incorporated by references for the subject matter and purpose referenced herein. In another example, the gene editing system (e.g., the CRISPR/Cas-mediated gene editing system) can comprise a gRNA targeting the AGGTTCCCTACACGGTCTCC (SEQ ID NO: 101) site in the CD83 gene.

TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

In some embodiments, an edited TRAC gene may comprise one or more nucleotide sequences selected from those listed in Table 18. It is known to those skilled in the art that different nucleotide sequences in an edited gene such as an edited TRAC gene (e.g., those in Table 18) may be generated by a single gRNA such as the one listed in Table 17 (TA-1). See, also WO2019097305, the relevant disclosures of which are incorporated by reference for the subject matter and purpose referenced herein.

β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

In some embodiments, an edited β2M gene may comprise one or more nucleotide sequence selected from those listed in Table 19. It is known to those skilled in the art that different nucleotide sequences in an edited gene such as an edited β2M gene (e.g., those in Table 19) may be generated by a single gRNA such as the ones listed in Table 17 (β2M-1 or β2M-4). See, also WO2019097305, the relevant disclosures of which are incorporated by reference for the subject matter and purpose referenced herein.

The genetically engineered T cells disclosed herein may further comprise one or more additional gene edits (e.g., gene knock-in or knock-out) to improve T cell function. Examples include knock-in or knock-out genes to improve target cell lysis, knock-in or knock-out genes to enhance performance of therapeutic T cells such as CAR-T cells prepared from the genetically engineered T cells.

It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.
(b) Exemplary Improved Features of Genetically Engineered T Cells Disclosed Herein

Any of the genetically engineered T cell having a disrupted Reg1 gene, a disrupted TGFBRII gene, optionally a disrupted CD83 gene, and optionally one or more additional genetic edits, for example, a disrupted TRAC gene, a disrupted β2M gene, a CAR-coding nucleic acid insertion, or a combination thereof, may be expandable in culture for greater than 4 weeks, for example, greater than 5 weeks, greater than 6 weeks, greater than 8 weeks, and greater than 10 weeks. In some examples, the genetically engineered T cells are expandable after 6 weeks (e.g., after 7 weeks, after 8 weeks, after 9 weeks, or after 10 weeks) in culture. Such genetically engineered T cells may maintain the ability to be activated after 6 weeks (e.g., after 7 weeks, after 8 weeks, after 9 weeks, or after 10 weeks) in culture. Further, such genetically engineered T cells have an increased expansion capacity, which can be at least 10-fold (e.g., at least 15-fold) higher than the non-engineered counterparts.

Further, the genetically engineered T cells disclosed herein may exhibit enhanced T cell persistence. “T cell persistence” as used herein refers to the tendency of T cells to continue to grow, proliferate, self-renew, expand, and maintain healthy activity in culture. In some instances, T cell persistence can be represented by the longevity that T cells can grow and expand in vitro, which can be measured by conventional methods and/or assays described herein. In other instances, T cell persistence can be represented by the reduction of cell death (e.g., apoptosis) or reduction in cell states characterized by exhaustion or replicative senescence. In yet other instances, T cell persistence can be presented by the maintenance of T cell activation capacity in culture.

Alternatively, or in addition, the genetically engineered T cells disclosed may grow faster and longer than the non-engineered T cells, for example, as observed in vitro cell culture. In some instances, the genetically engineered T cells may grow at least 50% (e.g., at least 1-fold, at least 2-fold, at least 5-fold, or more) than the non-engineered T cells in a conventional in vitro T cell culture (e.g., as described in Examples below). In other instances, the genetically engineered T cells may maintain a high growth rate (e.g., having substantially the same growth rate or with only a slight reduction) in vitro for at least 20 days (e.g., at least 25 days, at least 30 days, at least 35 days, at least 40 days, at least 45 days, at least 50 days, or longer).

In addition, the genetically engineered T cells may exhibit a reduced level of cell exhaustion as relative to the non-engineered T cell counterpart. In some instances, a reduced level of cell exhaustion is reflected by a higher level of central memory T cells in the whole T cell population. The population of genetically engineered T cells disclosed may comprise a higher number of central memory T cells as compared to non-engineered T cell counterparts. For example, in some instances the population of genetically engineered T cells include a higher number of central memory T cells that are characterized by enhanced expression of CD27 and/or CD45RO as compared to non-engineered T cell counterparts. In some instances, the population of genetically engineered T cells disclosed exhibit reduced T cell exhaustion, which is characterized, for example, by reduced expression of PD-1 and/or TIM3 as compared to non-engineered T cell counterparts.

Moreover, the genetically engineered T cells (e.g., CAR-T cells) may exhibit enhanced cytotoxicity activity, for example, against undesired cells (e.g., tumor cells) expressing an antigen targeted by the CAR expressed in the CAR-T cells, as compared with the non-engineered counterpart. Such genetically engineered T cells (e.g., CAR-T cells) may also be resistant to inhibitory effects mediated by the TGFI3 signaling and/or by fibroblast (e.g., in TME). For example, the genetically engineered T cells with a disrupted TGFBRII gene may be resistant to inhibitory factors secreted by fibroblasts.

CAR-T cells having triple disruptions of the CD83 gene, the TGFBRII gene, and the Reg1 gene were found to be more potent in cancer treatment than the counterpart T cells as observed in xenograft mouse models. Accordingly, CAR-T cells having at least the triple disruptions would be expected to show superior cancer treatment efficacy.

(c) Methods of Making Genetically Engineered T Cells

The genetically engineered T cells disclosed herein can be prepared by genetic editing of parent T cells or precursor cells thereof via a conventional gene editing method or those described herein.

(i) T Cells

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

(ii) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSB s) by specific rare-cutting nucleases (e.g., endonucleases). Such nuclease-dependent targeted editing also utilizes DNA repair mechanisms, for example, non-homologous end joining (NHEJ), which occurs in response to DSB s. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al., Vis. Exp. 2015; 95:e52118).

Available endonucleases capable of introducing specific and targeted DSB s include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxbl integrases may also be used for targeted integration. Some exemplary approaches are disclosed in detail below.

(a) CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

Endonuclease for Use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types Ito V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). Below is the amino acid sequence of Cas9 nuclease of S. pyogenes:

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of ordinary skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO:7), then the gRNA spacer sequence is 5′-AGAGCAACAGUGCUGUGGCC**-3′ (SEQ ID NO:4). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 17 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 17 below.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 5 of a TGFBRII gene, or a fragment thereof. Exemplary target sequences of TGFBRII and exemplary gRNA sequences are provided in Table 17 below.

In some embodiments, the gRNAs disclosed herein target a CD83 gene, for example, target a site within exon 2 or exon 3 of the CD83 gene. In some examples, the gRNA may target exon 2 of the CD83 gene. Such a gRNA may comprise a spacer sequence complementary (complete or partially) to the target sequences in exon 2 or exon 3 of a CD83 gene, or a fragment thereof. Exemplary target sequences in a CD83 gene and exemplary gRNA sequences are provided in Table 17 below. In one example, the gRNA targeting the CD83 gene can be CD83_exon2_G2 (a.k.a., CD83-2).

In some embodiments, the gRNAs disclosed herein target a β2M gene, for example, target a suitable site within a β2M gene. See, also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In some embodiments, the gRNAs disclosed herein target a TRAC gene. See, also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154;. Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein.

Exemplary spacer sequences and gRNAs targeting a β2M gene or TRAC gene are provided in Table 17 below. See, also WO2019097305, the relevant disclosures of which are incorporated by reference herein for the purpose and subject matter referenced herein.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed herein may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and an RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively, or in addition, a gRNA targeting a gene can be delivered to a cell as an RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used.

(b) Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion.

Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. No. 7,888,121 and U.S. Pat. No. 7,972,854. The most recognized example of a ZFN is a fusion of the Fokl nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the Fokl nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

### II. Genetically Engineered T Cells Expressing a Chimeric Antigen Receptor (CAR)

Disclosed herein are the genetically engineered T cells comprising a nucleic acid encoding a chimeric antigen receptor (CAR) that binds CD83 (anti-CD83 CAR), a disrupted Reg1 gene, a disrupted TGFBRII gene, or a combination thereof, and optionally a disrupted CD83 gene. Optionally, such genetically engineered T cells may comprise one or more of additional disrupted genes, e.g., β2M, TRAC, or a combination thereof as disclosed herein.

(a) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3t chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO:82 and SEQ ID NO:83 as provided in Table 22 below. Other signal peptides may be used.

(i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific CD83, which is a cell surface receptor on immune cells, for example, autoreactive immune cells or alloreactive immune cells, and on certain cancer cells such as leukemia cells.

In some embodiments, the antigen-binding extracellular domain can be a single-chain variable fragment (scFv) that binds a tumor antigen as disclosed herein. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively, the scFv may have the V_{L} to V_{H} orientation (from N-terminus to C-terminus).

In some examples, the antigen-binding extracellular domain can be a single-chain variable fragment (scFv) that binds human CD83. In some instances, the anti-CD83 scFv may comprises (i) a heavy chain variable region (V_{H}) that comprises the same heavy chain complementary determining regions (CDRs) as those in SEQ ID NO:77; and (ii) a light chain variable region (V_{L}) that comprises the same light chain CDRs as those in SEQ ID NO:78. In some specific examples, the anti-CD83 antibody discloses herein may comprise the heavy chain CDR1, heavy chain CDR2, and heavy chain CDR3 set forth as SEQ ID NOs: 71, 72 and 73, respectively, as determined by the Kabat method. Alternatively, or in addition, the anti-CD83 antibody discloses herein may comprise the light chain CDR1, light chain CDR2, and light chain CDR3 set forth as SEQ ID NOs: 74, 75 and 76, respectively, as determined by the Kabat method. In one specific example, the anti-CD83 scFv may comprise a V_{H} comprising the amino acid sequence of SEQ ID NO:77 and a V_{L} comprises the amino acid sequence of SEQ ID NO:78. See, Table 22 below. See, also U.S. Provisonal Application No. 63/303,666, the relevant disclosures thereof are incorporated by references for the subject matter and purpose referenced herein.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/ or abysis.org/abysis/sequence_input).

(ii) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of SEQ ID NO:87 as provided below in Table 22. Other transmembrane domains may be used.

(iii) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(iv) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of 30 CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 22 provides examples of signaling domains derived from 4-1BB, CD28 and CD3-zeta that may be used herein. In specific examples, the anti-CD83 CAR disclosed herein may comprise the amino acid sequence of SEQ ID NO:80 (with signal peptide) or SEQ ID NO:81 (without signal peptide), which may be encoded by the nucleotide sequence of SEQ ID NO:95. See, Tables 19 and 20 below.

(b) Delivery of CAR Construct to T Cells

In some embodiments, a nucleic acid encoding a CAR can be introduced into any of the genetically engineered T cells disclosed herein by methods known to those of skill in the art. For example, a coding sequence of the CAR may be cloned into a vector, which may be introduced into the genetically engineered T cells for expression of the CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a gene of interest to disrupt expression of the gene of interest. In some instances, the viral vector such as an adeno-associated viral (AAV) vector) can be designed such that it can insert into a location within the TRAC gene to disrupt the TRAC gene in the genetically engineered T cells and express the CAR polypeptide.

Disruption of TRAC leads to loss of function of the endogenous TCR. For example, a disruption in the TRAC gene can be created with an endonuclease such as those described herein and one or more gRNAs targeting one or more TRAC genomic regions. See, Table 17 below. Any of the gRNAs specific to a TRAC gene and the target regions disclosed herein can be used for this purpose.

In some embodiments, a disrupted gene of interest may comprise a deletion of a fragment, which may be the target site of a guide RNA used for making the disrupted gene. In some instances, the deleted fragment may be replaced by a donor template comprising the nucleotide sequence coding for the CAR polypeptide.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting a CAR coding segment into the TRAC gene.

In some embodiments, a nucleic acid encoding a CAR (e.g., via a donor template, which can be carried by a viral vector such as an adeno-associated viral (AAV) vector) can be designed such that it can insert into a location within a β2M gene to disrupt the β2M gene in the genetically engineered T cells and express the CAR polypeptide. Disruption of β2M leads to loss of function of the endogenous MHC Class I complexes. For example, a disruption in the β2M gene can be created with an endonuclease such as those described herein and one or more gRNAs targeting one or more β2M genomic regions. Any of the gRNAs specific to a β2M gene and the target regions disclosed herein can be used for this purpose.

In some examples, a genomic deletion in the β2M gene and replacement by a CAR coding segment can be created by homology directed repair or HDR (e.g., using a donor template, which may be part of a viral vector such as an adeno-associated viral (AAV) vector). In some embodiments, a disruption in the β2M gene can be created with an endonuclease as those disclosed herein and one or more gRNAs targeting one or more β2M genomic regions and inserting a CAR coding segment into the β2M gene.

In some embodiments, a nucleic acid encoding a CAR (e.g., via a donor template, which can be carried by a viral vector such as an adeno-associated viral (AAV) vector) can be designed such that it can insert into a location within a CD83 gene to disrupt the CD83 gene in the genetically engineered T cells and express the CAR polypeptide. Disruption of CD83 leads to loss of function of the endogenous CD83 protein. For example, a disruption in the CD83 gene can be created with an endonuclease such as those described herein and one or more gRNAs targeting one or more CD83 genomic regions. Any of the gRNAs specific to a CD83 gene and the target regions disclosed herein can be used for this purpose.

In some examples, a genomic deletion in the CD83 gene and replacement by a CAR coding segment can be created by homology directed repair or HDR (e.g., using a donor template, which may be part of a viral vector such as an adeno-associated viral (AAV) vector). In some embodiments, a disruption in the CD83 gene can be created with an endonuclease as those disclosed herein and one or more gRNAs targeting one or more Reg1 genomic regions and inserting a CAR coding segment into the CD83 gene.

In some embodiments, a nucleic acid encoding a CAR (e.g., via a donor template, which can be carried by a viral vector such as an adeno-associated viral (AAV) vector) can be designed such that it can insert into a location within a Reg1 gene to disrupt the Reg1 gene in the genetically engineered T cells and express the CAR polypeptide. Disruption of Reg1 leads to loss of function of the endogenous Regi protein. For example, a disruption in the Reg1 gene can be created with an endonuclease such as those described herein and one or more gRNAs targeting one or more Reg1 genomic regions. Any of the gRNAs specific to a Reg1 gene and the target regions disclosed herein can be used for this purpose.

In some examples, a genomic deletion in the Reg1 gene and replacement by a CAR coding segment can be created by homology directed repair or HDR (e.g., using a donor template, which may be part of a viral vector such as an adeno-associated viral (AAV) vector). In some embodiments, a disruption in the Reg1 gene can be created with an endonuclease as those disclosed herein and one or more gRNAs targeting one or more Reg1 genomic regions and inserting a CAR coding segment into the Reg1 gene.

In some embodiments, a nucleic acid encoding a CAR (e.g., via a donor template, which can be carried by a viral vector such as an adeno-associated viral (AAV) vector) can be designed such that it can insert into a location within a TGFBRII gene to disrupt the TGFBRII gene in the genetically engineered T cells and express the CAR polypeptide. Disruption of Reg1 leads to loss of function of the endogenous TGFBRII receptor. For example, a disruption in the TGFBRII gene can be created with an endonuclease such as those described herein and one or more gRNAs targeting one or more TGFBRII genomic regions. Any of the gRNAs specific to a TGFBRII gene and the target regions disclosed herein can be used for this purpose.

In some examples, a genomic deletion in the TGFBRII gene and replacement by a CAR coding segment can be created by homology directed repair or HDR (e.g., using a donor template, which may be part of a viral vector such as an adeno-associated viral (AAV) vector). In some embodiments, a disruption in the TGFBRII gene can be created with an endonuclease as those disclosed herein and one or more gRNAs targeting one or more TGFBRII genomic regions and inserting a CAR coding segment into the TGFBRII gene.

A donor template as disclosed herein can contain a coding sequence for a CAR. In some examples, the CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and 0-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO:94 provided in Table 23 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

When needed, additional gene editing (e.g., gene knock-in or knock-out) can be introduced into therapeutic T cells as disclosed herein to improve T cell function and therapeutic efficacy. For example, if β2M disruption can be performed to reduce the risk of or prevent a host-versus-graft response. Other examples include knock-in or knock-out genes to improve target cell lysis, knock-in or knock-out genes to enhance performance of therapeutic T cells such as CAR-T cells.

In some examples, a donor template for delivering an anti-CD83 CAR may be an AAV vector inserted with a nucleic acid fragment comprising the coding sequence of the anti-CD83 CAR, and optionally regulatory sequences for expression of the anti-CD83 CAR (e.g., a promoter such as the EF1α promoter provided in Table 23 below), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO:7. In some specific examples, the donor template for delivering the anti-CD83 CAR may comprise a nucleotide sequence of SEQ ID NO:95, which can be inserted into a disrupted TRAC gene, for example, replacing the fragment of SEQ ID NO:7.

The genetically engineered T cells having a disrupted Reg1 gene, additional disrupted genes, e.g., β2M and/or TRAC, and further expressing a chimeric antigen receptor (CAR) can be produced by sequential targeting of the genes of interest. For example, in some embodiments, the Reg1 gene may be disrupted first, followed by disruption of TRAC and β2M genes and CAR insertion. In other embodiments, TRAC and β2M genes may be disrupted first, followed by CAR insertion and disruption of the Reg1 gene. Accordingly, in some embodiments, the genetically engineered T cells disclosed herein may be produced by multiple, sequential electroporation events with multiple RNPs targeting the genes of interest, e.g., Reg1, β2M, TRAC, etc.

The genetically engineered T cells having a disrupted TGFBRII gene, additional disrupted genes, e.g., β2M and/or TRAC, and further expressing a chimeric antigen receptor (CAR) can be produced by sequential targeting of the genes of interest. For example, in some embodiments, the TGFBRII gene may be disrupted first, followed by disruption of TRAC and β2M genes and CAR insertion. In other embodiments, TRAC and β2M genes may be disrupted first, followed by CAR insertion and disruption of the TGFBRII gene. Accordingly, in some embodiments, the genetically engineered T cells disclosed herein may be produced by multiple, sequential electroporation events with multiple RNPs targeting the genes of interest, e.g., TGFBRII, β2M, TRAC, etc.

The genetically engineered T cells having a disrupted CD83 gene, additional disrupted genes, e.g., β2M and/or TRAC, and further expressing a chimeric antigen receptor (CAR) can be produced by sequential targeting of the genes of interest. For example, in some embodiments, the CD83 gene may be disrupted first, followed by disruption of TRAC and/or β2M genes and CAR insertion. In other embodiments, TRAC and/or β2M genes may be disrupted first, followed by CAR insertion and disruption of the CD83 gene. Accordingly, in some embodiments, the genetically engineered T cells disclosed herein may be produced by multiple, sequential electroporation events with multiple RNPs targeting the genes of interest, e.g., CD83 and optionally, β2M and/or TRAC.

In other embodiments, the genetically engineered CAR T cells disclosed herein may be produced by a single electroporation event with an RNP complex comprising an RNA-guided nuclease and multiple gRNAs targeting the genes of interest, e.g., Reg1, TGFBRII, CD83, β2M, and/or TRAC.
(c) Exemplary Genetically Engineered T Cells Expressing a Chimeric Antigen Receptor (CAR)

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell. For example, a cell having a CD83 gene edit may be considered a CD83 knockout cell if CD83 protein cannot be detected at the cell surface using an antibody that specifically binds CD83 protein.

In some embodiments, a population of genetically engineered immune cells such as T cells disclosed herein express a CAR (e.g., anti-CD83 CAR), a disrupted Reg1 gene, a disrupted TGFBRII gene, or a combination thereof, and optionally a disrupted CD83 gene. The genetically engineered T cells may further comprise a disrupted TRAC gene and/or a disrupted β2M gene. The nucleotide sequence encoding the CAR may be inserted in a genomic site of 5 interest, for example, in the disrupted TRAC gene (e.g., replacing the site targeted by a sgRNA such as TA-1 provided in Table 17 below), in the disrupted β2M gene (replacing the site targeted by a sgRNA such as β2M-1 provided in Table 17 below), in the disrupted CD83 gene (e.g., replacing the site targeted by a sgRNA such as those provided in Table 17 below), in the disrupted Reg1 gene (e.g., replacing the site targeted by the sgRNA listed in Table 17), or in the disrupted TGFBRII gene (e.g., replacing the site targeted by the sgRNA listed in Table 17)

In some examples, such a population of genetically engineered T cells may comprise at least 50% Reg1⁻ cells, for example, at least 60%, at least 70%, at least 80%, at least 90% or above, Reg1⁻ cells. In some examples, such a population of genetically engineered T cells may comprise at least 50% TGFBRII⁻ cells, for example, at least 60%, at least 70%, at least 80%, at least 90% or above, TGFBRII⁻ cells.

In some examples, such a population of genetically engineered T cells may comprise at least 50% CD83⁻ cells, for example, at least 60%, at least 70%, at least 80%, at least 90% or above, CD83⁻ cells. Alternatively, or in addition, the population of genetically engineered T cells may comprise at least 50% TCR⁻ cells, for example, at least 60%, at least 70%, at least 80%, at least 90% or above, TCR⁻ cells. Alternatively, or in addition, the population of genetically engineered T cells may comprise at least 50% β2M⁻ cells, for example, at least 60%, at least 70%, at least 80%, at least 90% or above, β2M⁻ cells. In some instances, the population of genetically engineered T cells may comprise at least 40% CAR+ cells (e.g., anti-CD83 CAR+ cells), for example, at least 50%, at least 60%, at least 70%, at least 80%, at least 90% or above, CAR+ cells.

In some examples, the population of genetically engineered T cells may comprise at least 50% of the engineered T cells expressing a detectable level of the CAR (e.g., an anti-CD83 CAR) and does not express a detectable level of CD83 on cell surface.

Any of the anti-CD83 CAR-T cells disclosed here may further express a second chimeric antigen receptor, which may be specific to a tumor antigen different from CD83. In some instances, the second chimeric antigen receptor can be a separate polypeptide relative to the anti-CD83 CAR. In other instances, the second chimeric antigen receptor may form a bi-specific CAR with the anti-CD83 CAR. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells. Exemplary tumor antigens include, but are not limited to, CD19, BCMA, CD70, and CD33.

(d) Anti-Inflammatory Agent

In some embodiments, any of the anti-CD83 CAR-T cells disclosed herein may be co-used with one or more anti-inflammatory agents. An anti-inflammatory agent as disclosed herein refers to any agent (e.g., polypeptide or protein) capable of suppressing inflammatory responses. In some embodiments, the anti-inflammatory agent may be an antagonist (e.g., antibody) of a pro-inflammatory cytokine (e.g., IL-1, IL-6, TNF-α, and B-cell activating factor (BAFF)). In other embodiments, the anti-inflammatory agent may be an agent that suppresses T cell co-activation, for example, an antibody or ligand that binds a T cell co-stimulatory receptor or a ligand thereof, thereby blocking the co-stimulation signaling pathway. In some embodiments, the anti-inflammatory agent may be a CTLA-4-Fc fusion polypeptide.

Cytotoxic T-lymphocyte associated protein 4 (CTLA-4), a.k.a., CD152, is a co-inhibitory molecule that plays a role in a negative feedback loop to reduce or dampen T-cell activation. It competes with CD28 for binding to CD80 orCD86, leading to suppression of T cell co-stimulation. CTLA-4 is a well-characterized protein. CTLA-4 proteins from various species can be found in public database, for example, GenBank. As one example, the structural information of human CTLA-4 can be found in GenBank under Gene ID: 1493 and the corresponding amino acid sequences of the corresponding gene transcript can be found, for example, under NM_005214.5 and NM_001037631.3.

The CTLA-4-Fc fusion polypeptide disclosed herein may comprise an extracellular domain of a CTLA-4 protein (e.g., human CTLA-4) fused to an Fc fragment of an antibody heavy chain constant region. The CTLA-4 portion of the CTLA-4-Fc fusion polypeptide disclosed herein may be from a wild-type CTLA-4 protein (e.g., a wild-type human CTLA-4 protein). Alternatively, the CTLA-4 portion may comprise one or more mutations relative to the wild-type counterpart to improve one or more desired features. For example, the CTLA-4 portion may comprise mutations as position A29, position L104, or a combination thereof. In some instances, the mutation is an amino acid residue substitution, for example, A29Y and/or L104E. Alternatively or in addition, the CTLA-4 poriton may comprise one or more conservative variations relative to the wild-type counterpart. In specific examples, the CTLA-4 portion in the CTLA-4-Fc fusion polypeptide disclosed herein is identical to the CTLA-4 portion in abatacept. In other specific examples, the CTLA-4 portion in the CTLA-4-Fc fusion polypeptide disclosed herein is identical to the CTLA-4 portion in belatacept.

The Fc fragment for use in making any of the CTLA-4-fusion polypeptide may be from any antibody subgroup, for example, IgG, IgA, IgE, IgD, or IgG, e.g., the hinge-CH2-CH3 domain of the antibody heavy chain. In some instances, the Fc fragment may be from a subfamily of IgG molecules, for example, IgG1 or IgG4. In some examples, the Fc fragment is of a wild-type immunoglobulin heavy chain. Alternatively, the Fc fragment may comprise one or more mutations relative to the wild-tye counterpart for modulating effector activities, and/or for reducing formation of undesired disulfide bonds. Such mutations may comprise mutations in the hinge domain (e.g., cysteine to surine substitutions).

### III. Therapeutic Applications

The therapeutic T cells generated using the genetically engineered T cells disclosed herein would be expected to maintain T cell health enabled by the disruption of the Reg1 gene, the disruption of the TGFBRII gene, and optionally the disruption of the CD83 gene, or a combination thereof. For example, maintaining T cell health may extend expansion during manufacturing, thereby increasing yield and consistency. In another example, maintaining T cell health may rescue exhausted/unhealthy T cells, thereby enabling potentially lower doses in patients and more robust responses. The anti-CD83 CAR-T cells disclosed herein would also be expected to reduce the risk of GvHD associated with CAR-T therapy.

The therapeutic T cells disclosed herein can be administered to a subject for therapeutic purposes, for example, treatment of an autoimmune disease or a cancer.

The step of administering may include the placement (e.g., transplantation) of the therapeutic T cells into a subject by a method or route that results in at least partial localization of the therapeutic T cells at a desired site, such as a tumor site, such that a desired effect(s) can be produced. Therapeutic T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment. For example, in some aspects described herein, an effective amount of the therapeutic T cells can be administered via a systemic route of administration, such as an intraperitoneal or intravenous route.

In some embodiments, the therapeutic T cells are administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human.

In some instances, the therapeutic T cells may be autologous (“self”) to the subject, i.e., the cells are from the same subject. Alternatively, the therapeutic T cells can be non-autologous (“non-self,” e.g., allogeneic, syngeneic or xenogeneic) to the subject. “Allogeneic” means that the therapeutic T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, an engineered T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient (e.g., subject). For example, an engineered T cell population, being administered to a subject can be derived from one or more unrelated donors, or from one or more non-identical siblings. In some embodiments, syngeneic cell populations may be used, such as those obtained from genetically identical donors, (e.g., identical twins). In some embodiments, the cells are autologous cells; that is, the engineered T cells are obtained or isolated from a subject and administered to the same subject, i.e., the donor and recipient are the same.

An effective amount refers to the amount of a population of engineered T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

Because of the enhanced persistence and efficacy of the therapeutic T cells disclosed herein, the dose of the therapeutic T cells provided herein would be lower than the standard dose of CAR-T cells prepared by conventional approaches (e.g., using T cells that do not have one or more of the genetic editing events disclosed herein, including a disrupted Reg1 gene, a disrupted TGFBRII gene and/or a disrupted CD83 gene). In some examples, the effective amount of the therapeutic T cells disclosed herein may be at least 2-fold lower, at least 5-fold lower, at least 10-fold lower, at least 20-fold lower, at least 50-fold lower, or at least 100-fold lower than a standard dose of a CAR-T therapy. In some examples, an effective amount of the therapeutic T cells disclosed herein may be less than 10⁶ cells, e.g., 10⁵ cells, 5×10⁴ cells, 10⁴ cells, 5×10³ cells, or 10³ cells. In some examples described herein, the cells are expanded in culture prior to administration to a subject in need thereof.

The efficacy of a treatment using the therapeutic T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the genetically engineered T cells disclosed herein can be used for eliminating undesired cells that are CD83+. In some examples, the undesired cells are cancer cells (e.g., CD83+ cancer cells). The genetically engineered T cells expressing an anti-CD83 CAR can be used for treating a CD83+ cancer. In other examples, the undesired cells are immune cells (e.g., CD83+ B cells or CD83+ dendritic cells). In some instances, the genetically engineered T cells expressing an anti-CD83 CAR can be used for treating an immune disorder, e.g., those in which the CD83+ immune cells play a role. The immune disorder may be an autoimmune disease, sepsis, rheumatological disease, diabetes, or asthma.

Any of the genetically engineered anti-CD83 CAR-T cells having a disrupted Reg1 and/or TGFBRII genes, optionally a disrupted CD83 gene, and optionally a disrupted TRAC and/or β2M gene can be administered to a subject for therapeutic purposes, for example, treatment of disease associated with CD83+ disease cells (e.g., cancer or an immune disease such as autoimmune disease). Exemplary immune diseases include lupus and chronic and acute GvHD. Exemplary cancer may be a hematopoietic cancer, for example, AML, CD19+ Leukemia, or CD19+ lymphomas. In some instances, a second population of CAR-T cells maybe co-used with the engineered immune cells having a disrupted CD83 gene as disclosed herein. For example, anti-CD19 CAR-T cells may be co-used with the CD83 disrupted immune cells for treating diseases involving CD19+ cells, such as AML, CD19+ Leukemia, or CD19+ lymphomas.

In some examples, the target disease can be a B cell mediated autoimmune disease. Examples include Examples include, but are not limited to, Achalasia, Acute disseminated encephalomyelitis (ADEM), Addison's disease, Adiposis dolorosa, Adult Still's disease, Agammaglobulinemia, Alopecia areata, Amyloidosis, Ankylosing spondylitis, Anti-GBM/Anti-TBM nephritis, Anti-N-Methyl-D-Aspartate (Anti-NMDA) receptor encephalitis, Antiphospholipid syndrome, Antisynthetase syndrome, Aplastic Anemia, Autoimmune angioedema, Autoimmune dysautonomia, Autoimmune encephalomyelitis, Autoimmune enteropathy, Autoimmune hepatitis, Autoimmune inner ear disease (AIED), Autoimmune lymphoproliferative syndrome, Autoimmune myocarditis, Autoimmune oophoritis, Autoimmune orchitis, Autoimmune pancreatitis, Autoimmune polyendocrine syndrome (APS) type 1, Autoimmune polyendocrine syndrome (APS) type 2, Autoimmune polyendocrine syndrome (APS) type 3, Autoimmune retinopathy, Autoimmune urticaria, Axonal & neuronal neuropathy (AMAN), Baló disease, Behcet's disease, Benign mucosal pemphigoid, Bickerstaff's encephalitis, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Drug-induced lupus, Endometriosis, Enthesitis-related arthritis, Eosinophilic esophagitis (EoE), Eosinophilic fasciitis, Epidermolysis bullosa acquisita, Erythema nodosum, Essential mixed cryoglobulinemia, Evans syndrome, Felty Syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hasimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgA Vasculitis, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus Nephritis, Lupus Vasculitis, Lyme disease chronic, Meniere's disease, Microscopic polyangiitis (MPA), Mixed connective tissue disease (MCTD), Mooren's ulcer, Morphea, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyotonia, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), Pityriasis lichenoides et varioliforis acuta, POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cholangitis, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Schnitzler syndrome, Scleritis, Scleroderma, Sjogren's syndrome, Sperm & testicular autoimmunity, Stiff person syndrome (SPS), Subacute bacterial endocarditis (SBE), Susac's syndrome, Sydenham's chorea, Sympathetic ophthalmia (SO), Systemic lupus erythematosus (SLE), Takayasu's arteritis, Temporal arteritis/Giant cell arteritis, Thrombocytopenia, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Urticarial vasculitis, Uveitis, Vasculitis, Vitiligo, and Vogt-Koyanagi-Harada Disease.

Combination therapies are also encompassed by the present disclosure. For example, the therapeutic T cells disclosed herein may be co-used with other therapeutic agents, for treating the same indication, or for enhancing efficacy of the therapeutic T cells and/or reducing side effects of the therapeutic T cells. In some instances, the anti-CD83 CAR-T cells disclosed herein may be in combined use with one or more additional CAR-T therapeutics targeting one or more tumor antigens such as those disclosed herein.

The clinical outcome of a treatment comprising a composition for the treatment of a medical condition can be determined by the skilled clinician. A treatment is considered effectiveif any one or all of the signs or symptoms of, as but one example, levels of functional target are altered in a beneficial manner (e.g., increased by at least 10%), or other clinically accepted symptoms or markers of disease (e.g., GvHD or an autoimmune disease) are improved or ameliorated. Clinical outcome can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, a method of suppressing immune responses in a subject is provided herein. This method comprises administering to a subject in need thereof (a) an effective amount of a population of genetically engineered T cells as described above and herein, and (b) an effective amount of an anti-inflammatory agent. In some instances, the population of genetically engineered T cells and the anti-inflammatory agent (e.g., an anti-CTLA-4-Fc fusion polypeptide such as belatacept) may be formulated in separate compositions, which may be administered to a subject in need of the treatment separately or together, concurrently or sequentially.

In any of the combination therapies disclosed herein, the anti-inflammatory agent is an antibody that inhibits a pro-inflammatory cytokine. Alternatively, the anti-inflammatory agent may be an agent that inhibits T-cell co-stimulation. In some embodiments, the anti-inflammatory agent is a CTLA4-Fc fusion protein. In some examples, the CTLA4-Fc fusion protein is belatacept. Alternatively, the CTLA-4-Fc fusion protein is abatacept. Any of the anti-inflammatory agents disclosed herein may comprise a signal peptide at its N-terminus. Non-limiting examples include a signal peptide of endogenous CTLA4 or a signal peptide of an immunoglobulin heavy chain.

In some embodimetns, the subject is a human patient. Such a human patient may have or at risk for an immune disease, e.g., an autoimmune disease as those disclosed herein or a graft-versus-host disease (GvHD). In some examples, the human patient has an autoimmune disease. In some examples, the human patient is at risk for or has GvHD. Such a human patient may have undergone or will be treated by an allogenic cell therapy, for example, an allogenic CAR-T cell therapy.

### IV. Kits

The present disclosure also provides kits for use in producing the genetically engineered T cells, the therapeutic T cells, and for therapeutic uses,

In some embodiments, a kit provided herein may comprise components for performing genetic edit of one or more of Reg1 gene, TGFBRII gene, and CD83 gene, and optionally a population of immune cells to which the genetic editing will be performed (e.g., a leukopak). A leukopak sample may be an enriched leukapheresis product collected from peripheral blood. It typically contains a variety of blood cells including monocytes, lymphocytes, platelets, plasma, and red cells. The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting a Reg1 gene, a TGFBRII gene, and/or a CD83 gene. Any of the gRNAs specific to these target genes can be included in the kit. Such a kit may further comprise components for further gene editing, for example, gRNAs and optionally additional endonucleases for editing other target genes such as β2M and/or TRAC.

In some embodiments, a kit provided herein may comprise a population of genetically engineered T cells as disclosed herein, and one or more components for producing the therapeutic T cells as also disclosed herein. Such components may comprise an endonuclease suitable for gene editing and a nucleic acid coding for a CAR construct of interest such as anti-CD83 CAR. The CAR-coding nucleic acid may be part of a donor template as disclosed herein, which may contain homologous arms flanking the CAR-coding sequence. In some instances, the donor template may be carried by a viral vector such as an AAV vector.

The kit may further comprise gRNAs specific to a TRAC gene for inserting the CAR-coding sequence into the TRAC gene. In other examples, the kit may further comprise gRNAs specific to a β2M gene for inserting the CAR-coding sequence into the β2M gene. In other examples, the kit may further comprise gRNAs specific to a CD83 gene for inserting the CAR-coding sequence into the CD83 gene. In yet other examples, the kit may further comprise gRNAs specific to a Reg1 gene for inserting the CAR-coding sequence into the Reg1 gene. In still other examples, the kit may further comprise gRNAs specific to a TGFBRII gene for inserting the CAR-coding sequence into the TGFBRII gene.

In yet other embodiments, the kit disclosed herein may comprise a population of therapeutic T cells as disclosed for the intended therapeutic purposes.

Any of the kit disclosed herein may further comprise instructions for making the therapeutic T cells, or therapeutic applications of the therapeutic T cells. In some examples, the included instructions may comprise a description of using the gene editing components to genetically engineer one or more of the target genes (e.g., Reg1, TGFBRII, CD93, or a combination thereof). In other examples, the included instructions may comprise a description of how to introduce a nucleic acid encoding a CAR construction into the T cells for making therapeutic T cells.

Alternatively, the kit may further comprise instructions for administration of the therapeutic T cells as disclosed herein to achieve the intended activity, e.g., eliminating disease cells targeted by the CAR expressed on the therapeutic T cells. The kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Some aspects of the embodiments discussed above are disclosed in further detail in the following examples, which are not in any way intended to limit the scope of the present disclosure.

### Example 1

### CAR T Cell Generation and CAR Expression

Allogeneic human T cells that lack expression of the TRAC gene, β2M gene, CD83 gene, TGFBRII gene and Regnase-1 gene, and express a chimeric antigen receptor (CAR) targeting CD83 were produced. Relative to T cell counterparts having a wild-type CD83 gene, such genetically engineered immune cells showed enhanced overall T cell expansion, reduced levels of pro-inflammatory cytokine production, and increased CD8+ cell frequency with no negative impact on CAR-T cell function.

CD83 knockout was introduced into the 1st and 2′ generation IO CAR-T chassis. Both generations of the CAR-T cells lacked expression of the TRAC gene and β2M gene. See, Table 1 below.

TABLE 1
CAR-T Cells with CD83 Knockout (KO)

| | TRAC | β2M | CD83 | CD83 | TGFBRII | Regnase |
|---|---|---|---|---|---|---|
| Cells | KO | KO | CAR | KO | KO | KO |
| CD83 CAR: 83 KO | Yes | Yes | Yes | Yes | No | No |
| (1^{st} generation) | | | | | | |
| CD83 CAR: 83 + | Yes | Yes | Yes | Yes | Yes | Yes |
| R + T KO | | | | | | |
| (2^{nd} generation) | | | | | | |

Briefly, PBMCs were thawed and activated with TransAct. After 0-3 days, the cells were electroporated with Cas9:sgRNA RNP complexes and transduced with adeno-associated adenoviral vectors (AAVs) to generate anti-CD83 CAR T cells with CD83 KO (denoted as CD83 CAR+83 KO) or anti-CD83 CAR T cells with CD83, Regnase-1, and TGFBRII knockouts (denoted as CD83 CAR+83 KO+R/T KO). The sgRNAs, which form RNPs with the Cas9 enzyme, can be introduced into the T cells in a single or multiple electroporation events. After the electroporation, the cells were transduced with the recombinant AAVs to introduce the donor template encoding the anti-CD83 CAR. Recombinant AAV serotype 6 (AAV6) comprising one of the nucleotide sequences encoding an anti-CD83 CAR (SEQ ID NO:95) was delivered with Cas9:sgRNA RNPs (1 μM Cas9, 5 gRNA) to activated allogeneic human T cells. The following sgRNAs were used: CD83 (SEQ ID NO: 20 or 21); TRAC (SEQ ID NO: 2 or 3), β2M (SEQ ID NO: 8, 9, 14, or 15), TGFBRII (SEQ ID NO: 26 or 27) and REG-1 (SEQ ID NO: 32 or 33). The unmodified versions (or other modified versions) of the sgRNAs may also be used. See, the representative gRNA sequences provided in Table 17 below, including both unmodified and modified versions.

Assessment of T-Cell Expansion

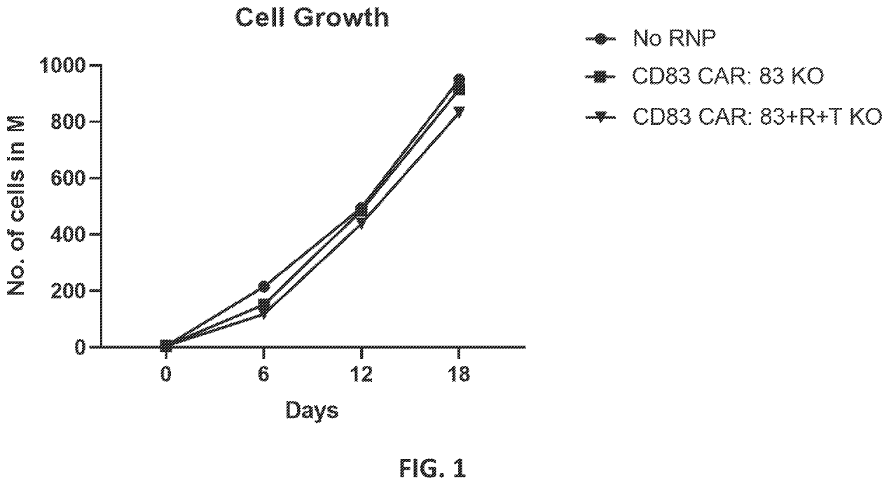

The cells were counted at regular intervals to assess T-cell expansion. The results are presented in FIG. 1 and Table 2 below. The data demonstrated that there was no impact on cell growth with the Regnase and TGFBRII knock-out edits. Edited cells expanded at a similar rate to non-edited T-cells.

TABLE 2
T-cell Number

| | T-Cell Numbers (×10⁶) | | | |
|---|---|---|---|---|
| Cell Name | Day 0 | Day 6 | Day 12 | Day 18 |
| Control cells (No RNP) | 5 | 216 | 495 | 951 |
| CD83 CAR: 83 KO | 5 | 152 | 485 | 915 |
| CD83 CAR: 83 + R + T KO | 5 | 118 | 439 | 833 |

### Assessment of Editing Efficiency

CAR expression was also measured at regular intervals. CAR detection was done by using 1-10 μg/mL of His tagged CD83 antigen followed by anti-His APC conjugated antibody, as well as by ddPCR. The flow cytometry results are presented as the average of two donors and the ddPCR are the mean copy number of 3 replicated in Table 3 below. There was no significant difference in CAR expression on KO of Regnase and TGFBRII in CAR T cells with CD83 KO.

TABLE 3
CAR+ Cells

CAR+ Cell (% population)

| Cell Name | By flow | By ddPCR |
|---|---|---|
| CD83 CAR: 83 KO | 21 | 0.97 |
| CD83 CAR: 83 + R + T KO | 20 | 0.86 |

Editing efficiency for TRAC, β2M, CD83, TGFBRII, and Regnase knockouts were also assessed by flow cytometry and TIDE analysis. The results are presented in Table 4 below. Similar levels of editing efficiency for TRAC, β2M and CD83 were observed in the presence or absence of Regnase and TGFBRII.

TABLE 4
Editing Efficiency

| | Editing Efficiency (%) | | | | |
|---|---|---|---|---|---|
| | TRAC | β2M | CD83 | TGFBRII | Regnase |
| | KO | KO | KO | KO | KO |
| Sample | (flow) | (flow) | (TIDE) | (TIDE) | (TIDE) |
| CD83 CAR: 83 | 97 | 97 | 95 | N/A | N/A |
| KO | | | | | |
| CD83 CAR: | 94 | 93 | 96 | 81 | 92 |
| 83 + R + T KO | | | | | |

Assessment of CD4: CD8 Ratios

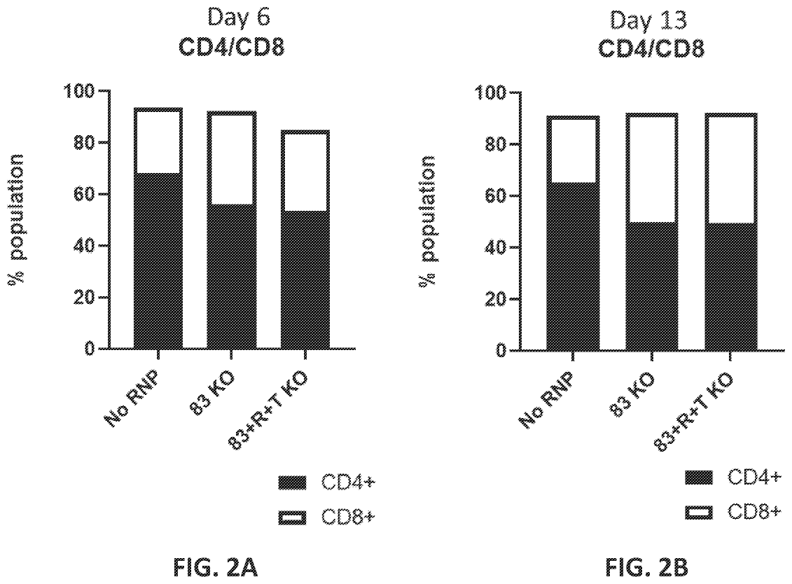

The frequency of CD4 and CD8 T cells in these cell cohorts was also determined by flow cytometry on Day 6 and Day 13. Average frequencies are enumerated in FIGS. 2A-2B and Table 5 below. There was no significant impact on CD4+ to CD8+ cell ratios in the cell populations with the Regnase and TGFBRII gene edits.

TABLE 5
CD4+ and CD8+ Cell Percentages

% Population

| Cell Name | CD4+ | CD8+ |
|---|---|---|
| Control cells (No RNP) | 62 | 31 |
| CD83 CAR: 83 KO | 60 | 34 |
| CD83 CAR: 83 + R + T KO | 52 | 37 |

### Assessment of Immune Checkpoint Molecules

The expression of two immune checkpoint molecules, PD1 and Lag3 were evaluated in the cell populations. PD1 as an immune checkpoint molecule down-regulates T cell activity during immune responses to prevent autoimmune tissue damage (Jubel et al., Front. Immunol. 2020). Lymphocyte activation gene-3 (LAG-3) is an important immune checkpoint with relevance in cancer, infectious disease, and autoimmunity (Graydon et al., Front. Immunol. 2021). As a coinhibitory immune checkpoint, LAG3 inhibits the activation of its host cell and generally promotes a more suppressive immune response.

The expression levels were measured on Day 13 and Day 24 and are presented in Tables 6-7 below. The data demonstrates that there was no significant change in PD1 and Lag3 levels between anti-CD83 CAR T cells with or without Regnase and TGFBRII editing. PD-1 and Lag3 levels dropped between Day 13 and 24 for both genotypes tested.

TABLE 6
Level of PD1+ Cells

PD1⁺ Cell (% population)

| Cell Name | Day 13 | Day 24 |
|---|---|---|
| Control cells (No RNP) | 10 | 1.7 |
| CD83 CAR: 83 KO | 5 | 1.3 |
| CD83 CAR: 83 + R + T KO | 4 | 0.8 |

TABLE 7
Level of Lag3+ Cells

Lag3⁺ Cell (% population)

| Cell Name | Day 13 | Day 24 |
|---|---|---|
| Control cells (No RNP) | 14 | 7 |
| CD83 CAR: 83 KO | 16 | 5 |
| CD83 CAR: 83 + R + T KO | 18 | 5 |

In sum, the results provided in this example indicate that genetically disrupting the Reg1 gene and TGFBRII gene in anti-CD83 CAR-T cells with disrupted TRAC, β2M, and CD83 genes showed no signicant impact on CAR-T cell properties, e.g., T-cell growth, gene editing efficiency, CD4:CD8 ratio, and levels of immune checkpoint markers.

### Example 2

### In Vitro Cytotoxicity of Anti-CD83 CAR T Cells

Cell killing (cytotoxicity) assays were used to assess the ability of the CAR^{'} T cell populations described in Example 1 to cause cellular lysis in A498 cancer cell lines. An in votro re-challenge assay was performed as follows.

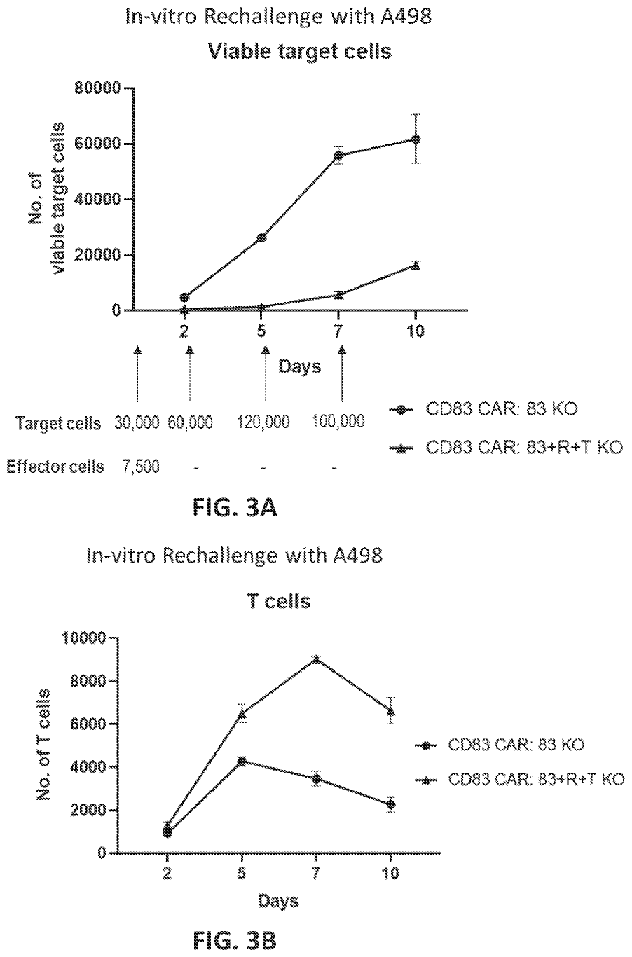

CAR T cells (7,500 cells per well) were plated with 30,0000 target cells. On Days 2, 5, and 7 of incubation, additional A498 cells at 60,000 cells, 120,000 cells and 100,000 cells were added to the culture. The numbers of viable target cells and T cells was counted and are presented in FIGS. 3A-3B and Tables 8-9 below. The data demonstrates that there was significantly improved target cell killing, as well as increased expansion when activated by target cells, of CAR T cells with Regnase and TGFBRII edits.

TABLE 8
Target Cell Numbers

| Cell Name | Day 2 | Day 5 | Day 7 | Day 10 |
|---|---|---|---|---|
| CD83 CAR: 83 KO | 4,694 | 26,145 | 55,766 | 61,725 |
| CD83 CAR: 83 + R + T KO | 529 | 1,321 | 5,665 | 16,273 |

TABLE 9
CAR T-Cell Numbers

| Cell Name | Day 2 | Day 5 | Day 7 | Day 10 |
|---|---|---|---|---|
| CD83 CAR: 83 KO | 916 | 4,270 | 3,475 | 2,263 |
| CD83 CAR: 83 + R + T KO | 1,280 | 6,498 | 9,026 | 6,628 |

### Example 3

### In Vivo Cytotoxicity of Anti-CD83 CART Cells

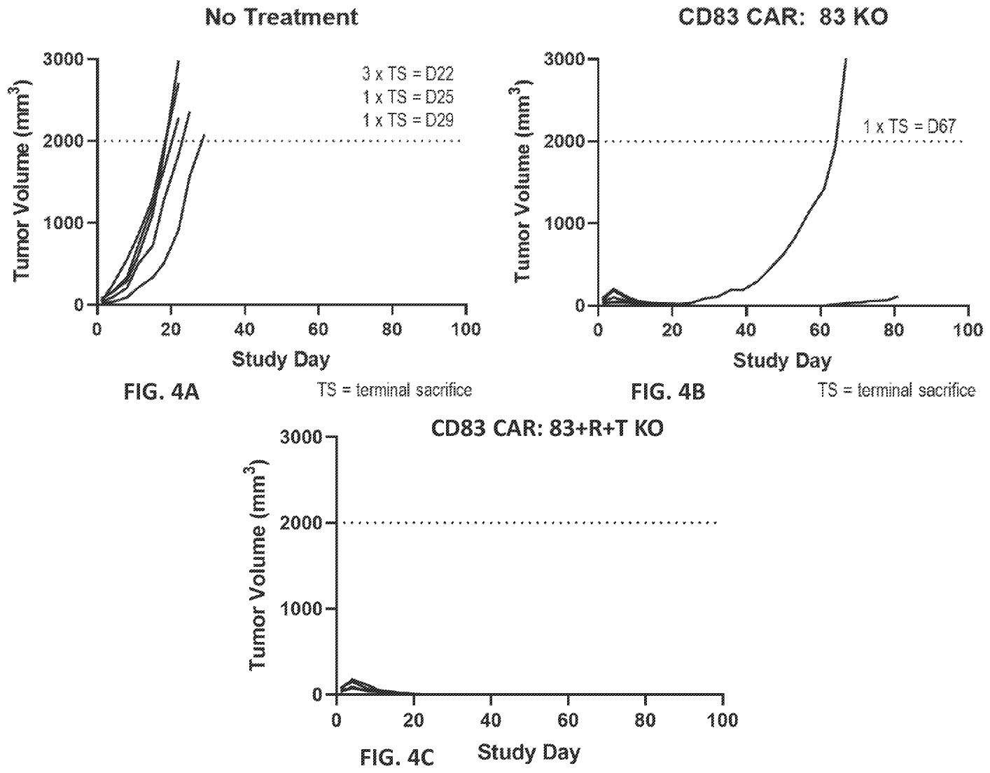

The CAR T cells generated as described in Example 1 were evaluated in an established THP-1 human acute monocytic leukemia xenograft model in NSG mice. Female NSG mice were subcutaneously implanted on the right flank with 5×10⁶ tumor cells in 50% Matrigel/50% media. Ten (10) days later, when tumor volume was ˜50 mm3 (range 25-75 mm³), the mice were randomized into 5 groups and injected intravenously with CAR T cells at 107 CAR+ cells per mouse. The groups are shown in FIGS. 4A-4C and Table 10 below.

Tumor volumes were evaluated every few days. Tumor volumes are presented in Table 10. Survival of the mice was also evaluated and is presented in Table 10. ‘N/A’ indicates that there were no surviving mice at that time point. The data demonstrates that knockout of Regnase and TGFBRII in CD83 CAR cells with CD83 KO significantly enhanced the anti-tumor activity.

TABLE 10
Tumor Volume

| | Tumor Volumes (mm³) | | | | | | | | | | | | | | | |
|---|---|---|---|---|---|---|---|---|---|---|---|---|---|---|---|---|
| Cell | Day | Day | Day | Day | Day | Day | Day | Day | Day | Day | Day | Day | Day | Day | Day | Day |
| Name | 1 | 8 | 15 | 18 | 22 | 25 | 29 | 36 | 43 | 50 | 61 | 67 | 71 | 81 | 88 | 95 |
| Untreated | 53 | 297 | 937 | 1437 | 2147 | 1965 | 2081 | N/A | N/A | N/A | N/A | N/A | N/A | N/A | N/A | N/A |
| CD83 | 53 | 67 | 13 | 9 | 6 | 7 | 18 | 39 | 60 | 125 | 285 | 611 | 10 | 29 | 0 | 0 |
| CAR: 83 | | | | | | | | | | | | | | | | |
| KO | | | | | | | | | | | | | | | | |
| CD83 | 52 | 62 | 14 | 3 | 0 | 0 | 0 | 0 | 0 | 0 | 0 | 0 | 0 | 0 | 0 | 0 |
| CAR: | | | | | | | | | | | | | | | | |
| 83 + R + T | | | | | | | | | | | | | | | | |
| KO | | | | | | | | | | | | | | | | |

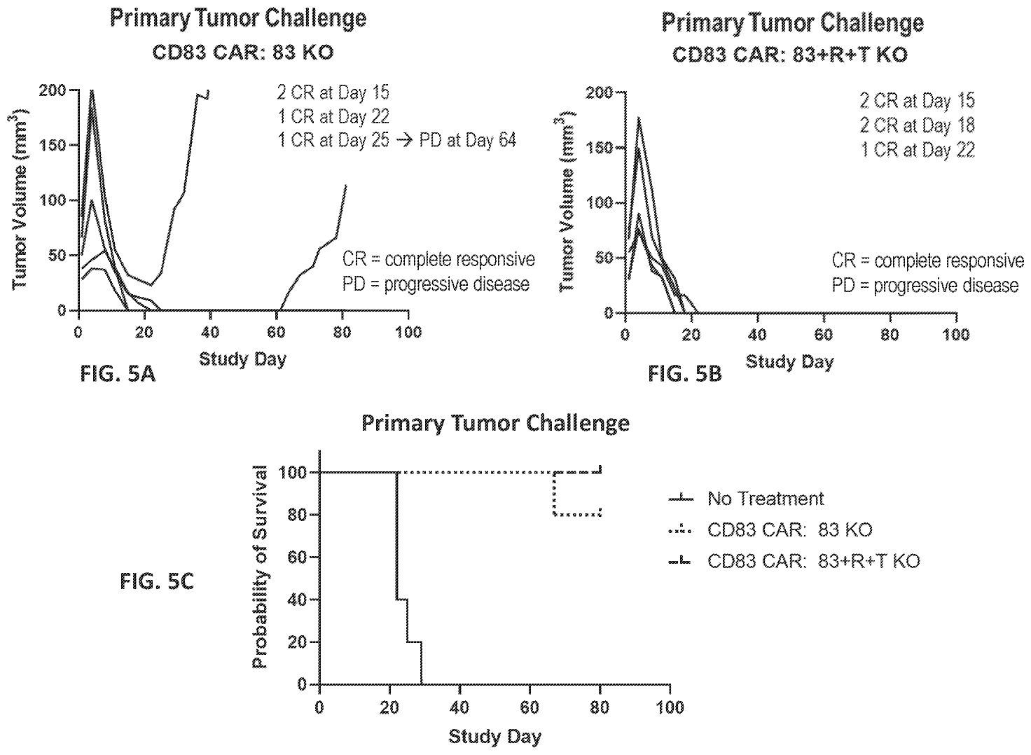

Survival of the mice is shown in FIGS. 5A-5C and Table 11 below and confirms that knockout of CD83, Regnase and TGFBRII in these cells significantly increases the anti-tumor activity with complete response from the primary tumor challenge seen in 2 of 5 mice 5 in the CD83 CAR+83 KO+R/T KO population.

TABLE 11
Median Survival

| Cell Name | Days |
|---|---|
| Untreated | 22 |
| CD83 CAR: 83 KO | Not reached |
| CD83 CAR: 83 + R + T KO | Not reached |

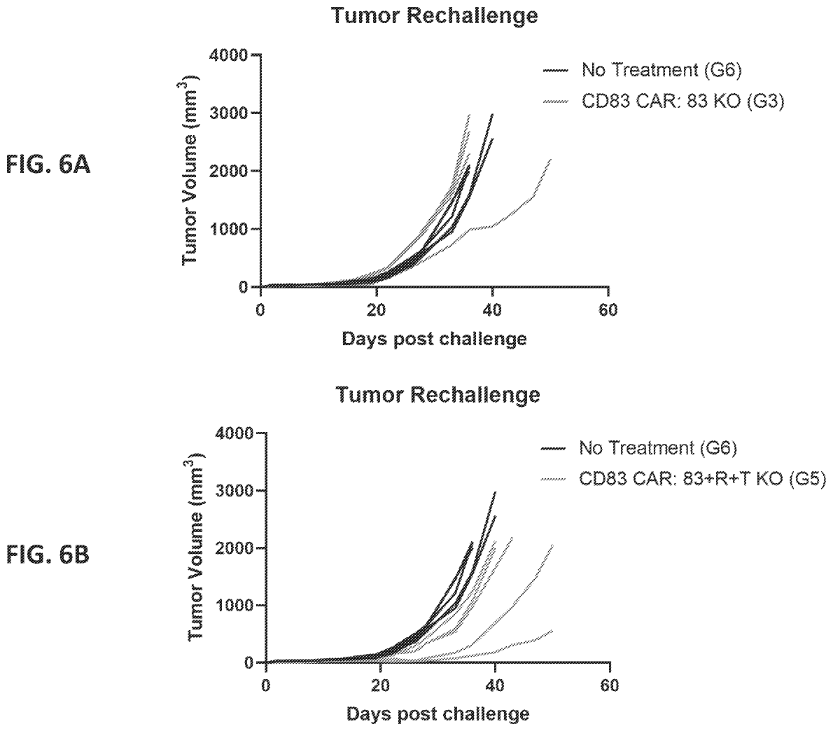

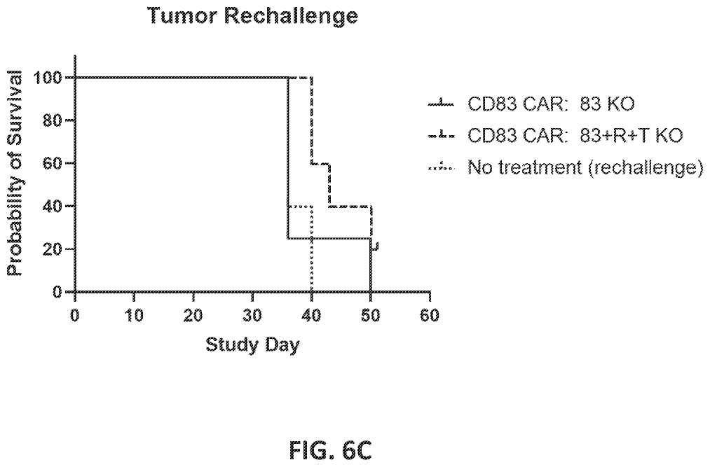

On Day 45 after test article administration, mice from Groups 3 (CD83 CAR: 83 KO) and 5 (CD83 CAR: 83 KO +R/T KO) were rechallenged with 5×10⁶ THP-1 tumor cells implanted on the left flank. A control group of mice was also implanted with the tumor and did not receive any CAR T cell inoculation. Tumor volumes and survival are shown in FIGS. 6A-6C and Tables 12-13 below, which confirmed that knockout of the Reg1 and TGFBRII genes enhances activity of anti-CD83 CAR T cells with CD83 KO.

TABLE 12
Tumor Volume after Rechallenge

| | Tumor Volumes (mm³) | | | | | | | | |
|---|---|---|---|---|---|---|---|---|---|
| | Day | Day | Day | Day | Day | Day | Day | Day | Day |
| Cell Name | 5 | 12 | 19 | 26 | 33 | 36 | 40 | 43 | 50 |
| Untreated | 30 | 53 | 117 | 426 | 1225 | 1870 | N/A | N/A | N/A |
| CD83 CAR: | 26 | 63 | 167 | 628 | 1432 | 2240 | 1045 | 1237 | 2208 |
| 83 KO | | | | | | | | | |
| CD83 CAR: | 22 | 45 | 67 | 153 | 448 | 748 | 1334 | 1161 | 1306 |
| 83 + R + T KO | | | | | | | | | |

TABLE 13
Median Survival

| Cell Name | Days |
|---|---|
| Untreated | 36 |
| CD83 CAR: 83 KO | 36 |
| CD83 CAR: 83 + R + T KO | 43 |

In sum, the results provided in this example demonstrate that genetic disruption of the Reg1 and TGFBRII genes significantly enhanced the in vivo anti-tumor activity of anti-CD83 CAR-T cells with disrupted TRAC, β2M, and CD83 genes as observed in an animal model. Accordingly, anti-CD83 CAR-T cells with disrupted Reg1 and TGFBRII genes, and optionally with disrupted TRAC, β2M, and CD83 genes, would be expected to exhibit superior therapeutic efficacy of diseases involving CD83, e.g., CD83+ tumor.

### Example 4

### Impact of CD83, Regnase and TGFBRII Knockout on Activity of CD83 CAR T Cells In Vivo

This example assesses the effect of disrupting the CD83, Regnase and TGFBRII genes in CD83 CAR T cells in vivo using a xenogeneic graft versus host disease (GvHD) model in mice as well as an established THP-1 xenograft tumor in mice.

Preventative GvHD model study

NSG mice were individually housed in ventilated microisolator cages, maintained under pathogen-free conditions, 5-7 days prior to the start of the study. The mice each received a subcutaneous inoculation of 20×10⁶ PBMCs/mouse to induce GvHD. The mice were further divided into 4 treatment groups and treatment groups 2 to 5 were co-administered a single intravenous dose of T cells. The CAR T cells were made from a donor that was allogeneic to the PBMC donor used to humanize the NSG mice. The CD83_exon2_3 gRNA (see Table 17 below) was used in this study to disrupt the CD83 gene.

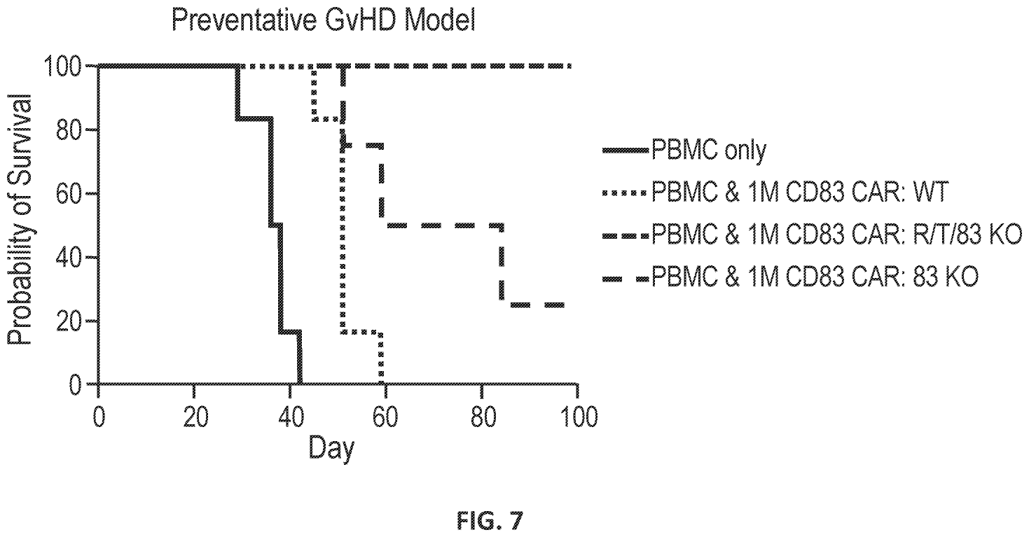

Survival of the mice groups is shown in FIG. 7. The CAR-T cells used in this study were allogeneic to the PBMC donor. It was observed that anti-CD83 CAR 83/R/T KO cells prevented GvHD at 1M CAR+ dose levels until at least Day 99, while anti-CD83 CAR 83 KO cells delayed GvHD at the 1M CAR+ dose level until Day 84. As shown in FIG. 7, none of the mice injected with PBMC alone survive past Day 42 while mice injected with PBMC and allogeneic anti-CD83 CAR T cells survive until Day 59. The results show that disrupting the CD83, Regnase-1, and TGFBRII genes significantly enhanced the activity of anti-CD83 CAR-T cells in a preventative GvHD model. This is consistent with data showing that CD83 CAR T cells subjected to fratricide during expansion exhibit an exhausted phenotype that reduces their potency in vitro and in vivo.

### Established Tumor Xenograft Model Study

The CAR T cells were evaluated in an established THP-1 human acute monocytic leukemia xenograft model in NSG mice. Female NSG mice were subcutaneously implanted on the right flank with 5×10⁶ tumor cells in 50% Matrigel/50% media. Ten days later, when tumor volume was ˜50 mm³ (range 25-75 mm3), the mice were randomized into 5 groups and injected intravenously with CAR T cells at 107 CAR+ cells per mouse.

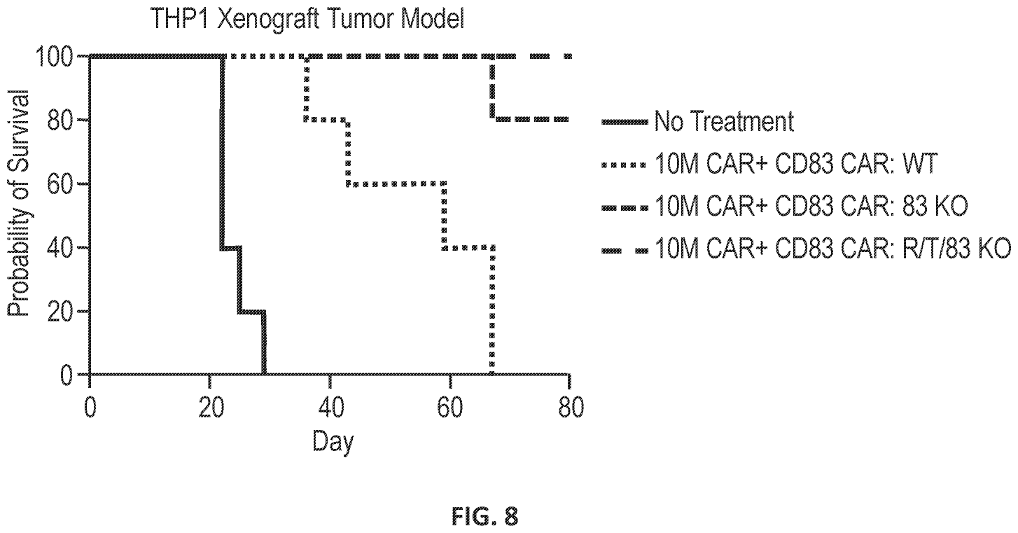

Survival of the mice was also evaluated and is presented in FIG. 8. Tumor-bearing control mice did not survive beyond Day 29 while mice treated with allogeneic anti-CD83 CAR T cells survived until Day 67. Only one mouse from the anti-CD83 CAR T cells with CD83 KO died on day 67 while all the mice treated with anti-CD83 CAR T cells with CD83, Regnase-1, and TGFBRII knockouts survived at least until Day 80. The results show that knockout of CD83, Regnase-1, and TGFBRII genes significantly enhanced the anti-tumor activity of anti-CD83 CAR T cells against THP-1 xenograft tumors.

### Example 5

### Impact of Belatacept Treatment on Activity of CD83 CAR T Cells In Vivo

This example explores the in vivo effects of the combination of belatacept and anti-CD83 CAR-T cell as proof of concept for arming anti-CD83 CAR-T cells with an anti-inflammatory agent payload (e.g., CAR-T cells expressing belatacept).

NSG mice were individually housed in ventilated microisolator cages, maintained under pathogen-free conditions, 5-7 days prior to the start of the study. Each mouse received a intravenous inoculation of 20×10⁶ PBMCs/mouse to induce GvHD. The mice were divided into 4 groups. Treatment groups #2-4 were co-administered with either a single intravenous dose of T cells, multiple intraperitoneal doses of belatacept (12×100 μg doses 3 times a week for 4 weeks), or both (Table 14). The CAR T cells were made from a donor that is allogeneic to the PBMC donor used to humanize the NSG mice. Information regarding the anti-CD83 CAR-T cells with a disrupted CD83 gene (CD83 knockout, using CD83_exon2_3 gRNA as an example) can be found in U.S. patent application Ser. No. 63/254,332, the relevant disclosures of which are incorporated by reference for the subject matter and purpose referenced herein.

TABLE 14
Treatment Groups

| | | PBMCs | CAR-T | Belatacept | |
|---|---|---|---|---|---|
| Group | Group Name | (IV) | (single dose) | (repeat dosing) | N |
| 1 | No treatment | 20 × 10⁶ | — | — | 6 |
| 2 | CD83 CAR + | 20 × 10⁶ | 1M CD83 | — | 6 |
| | CD83KO | | CAR T | | |
| 3 | Bela | 20 × 10⁶ | — | 12 × 100 μg | 4 |
| | | | | 3 × week for 4 | |
| | | | | weeks | |
| 4 | CD83 CAR + | 20 × 10⁶ | 1M CD83 | 12 × 100 μg | 4 |
| | CD83KO + | | CAR T | 3 × week for 4 | |
| | Bela | | | weeks | |

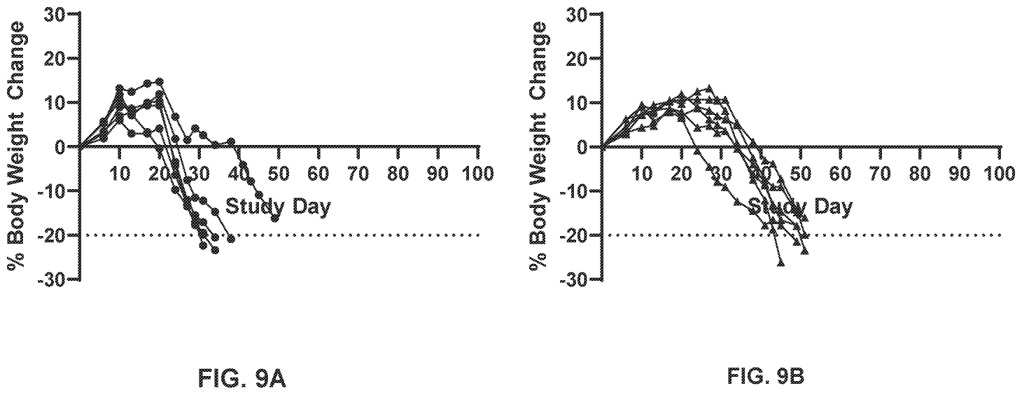

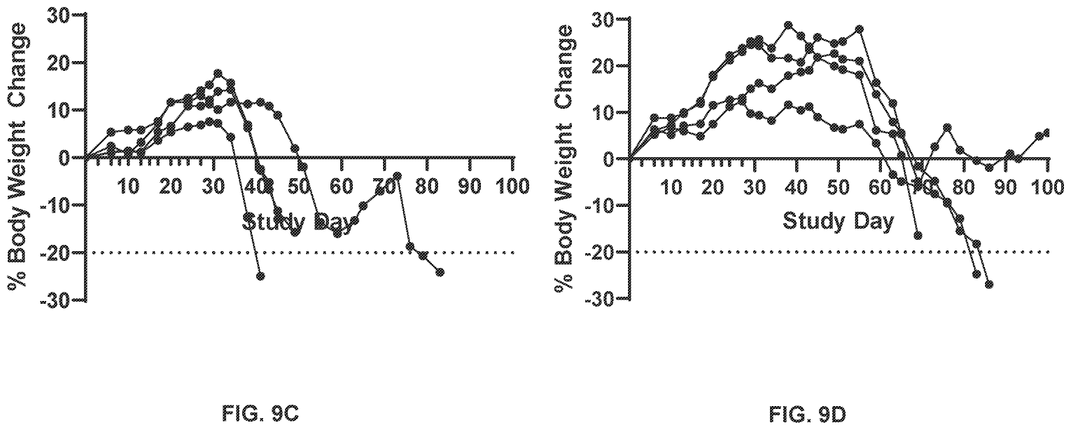

Body weights of the mice were measured as an indicator for development of graft-versus-host diseases (GvHD) at regular intervals and mice are euthanized when body weight loss reached 20% or more of the starting body weight. The data presented in the Table 15 shows that CD83 CAR T cells and Belatacept delayed the onset of xenogeneic GvHD, which is an improvement over individual treatment alone. N/A indicates that there were no available mice for body weight measurement. See also FIGS. 9A-9D.

TABLE 15
Body Weight Changes (% over baseline)

| | | Day | Day | Day | Day | Day | Day | Day | Day | Day | Day | Day |
|---|---|---|---|---|---|---|---|---|---|---|---|---|
| Group | Group Name | 6 | 10 | 20 | 31 | 43 | 55 | 65 | 73 | 83 | 91 | 100 |
| 1 | No treatment | 4 | 10 | 8 | −14 | −8 | N/A | N/A | N/A | N/A | N/A | N/A |
| 2 | CD83 | 4 | 8 | 9 | 4 | −12 | N/A | N/A | N/A | N/A | N/A | N/A |
| | CAR + CD83 | | | | | | | | | | | |
| | KO | | | | | | | | | | | |
| 3 | Bela | 3 | 2 | 9 | 12 | −0.4 | −14* | −10* | −4* | −24* | N/A | N/A |
| 4 | CD83 | 7 | 7 | 14 | 19 | 19 | 19 | 2 | −3 | −14 | 1* | 6* |
| | CAR + CD83 | | | | | | | | | | | |
| | KO + Bela | | | | | | | | | | | |

*one mouse survival

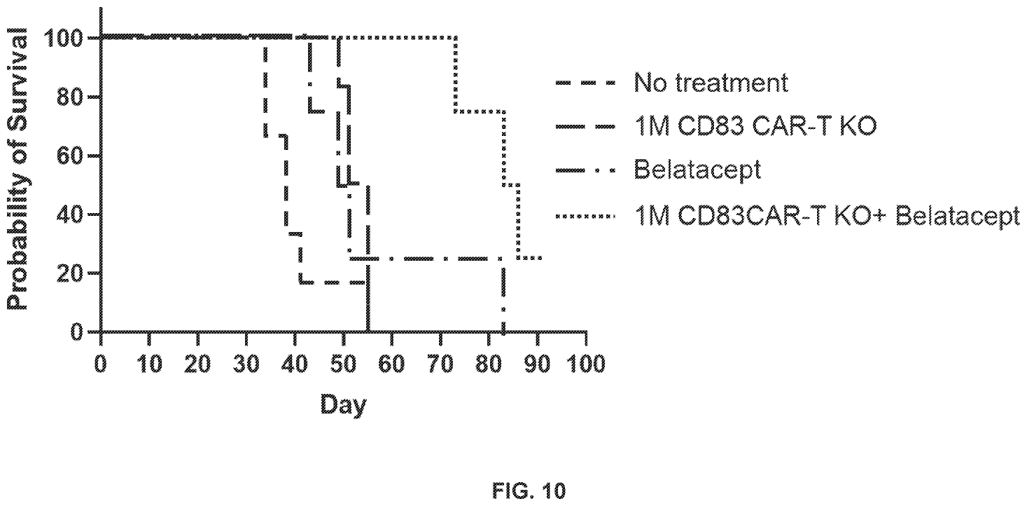

The impact of combining a CD83targeting CAR T with CD83KO and Belatacept is also evident from comparing the median survival time of mice in each treatment group (Table 16 and FIG. 10). Treatment with a CD83 CAR T with CD83 gene KO extended median survival time from 38 days to 53 days whereas the combination extended median survival time to 85 days.

TABLE 16
Survival Rates

| | | | Median | p value (no |
|---|---|---|---|---|
| | | Number | survival | treatment versus |
| Group | Group Name | of mice | (days) | other groups) |
| 1 | No treatment | 6 | 38 | — |
| 2 | CD83 CAR + | 6 | 53 | 0.0374 * |
| | CD83KO | | | |
| 3 | Belatacept | 4 | 50 | 0.1129 ns |
| 4 | CD83 CAR + | 4 | 85 | 0.0033 ** |
| | CD83KO + | | | |
| | Belatacept | | | |

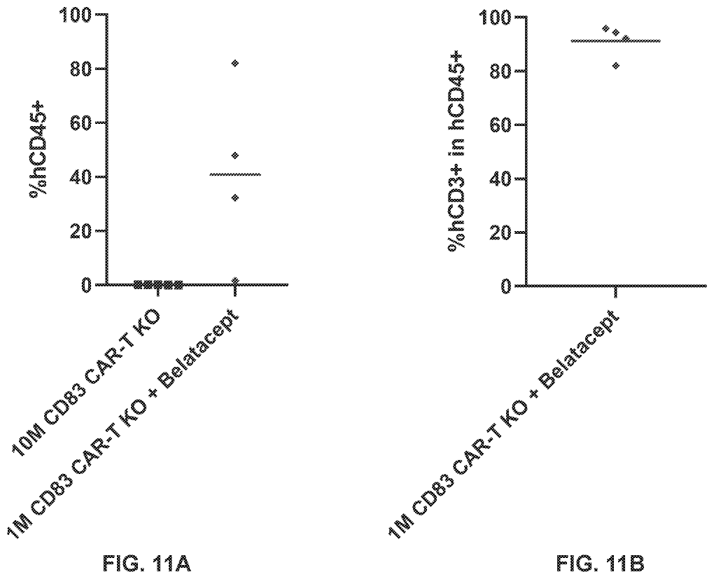

Analysis of engraftment levels on day 56 revealed the presence of circulating T-cells in the belatacept+CD83 CAR T combination group (FIGS. 11A and 11B). Human CD45 engraftment of high dose CD83 CAR-T KO group remained low, while human CD45+ cells detectable in the combination group were mostly T cells.

### Sequence Tables

TABLE 17
sRNA Sequences and Target Sequences

| | | | Target Sequence |
|---|---|---|---|
| Name | Unmodified Sequence | Modified Sequence | (PAM) |
| TRAC | AGAGCAACAGUGCUGUGGC | A*G*A*GCAACAGUGCUG | AGAGCAACAGTGCTGTG |
| sgRNA (TA- | Cguuuuagagcuagaaaua | UGGCCguuuuagagcuag | GCC (TGG) (SEQ ID |
| 1) | gcaaguuaaaauaaggcua | aaauagcaaguuaaaaua | NO: 6) |
| | guccguuaucaacuugaaa | aggcuaguccguuaucaa | AGAGCAACAGTGCTGTG |
| | aaguggcaccgagucggug | cuugaaaaaguggcaccg | GCC (SEQ ID NO: |
| | CUUUU | agucggugcU*U*U*U | 7) |
| | (SEQ ID NO: 2) | (SEQ ID NO: 3) | |
| TRAC | AGAGCAACAGUGCUGUGGC | A*G*A*GCAACAGUGCUG | |
| sgRNA | C (SEQ ID NO: 4) | UGGCC (SEQ ID NO: | |
| spacer | | 5) | |
| β2M-1 | GCUACUCUCUCUUUCUGGC | G*C*U*ACUCUCUCUUUC | GCTACTCTCTCTTTCTG |
| sgRNA | Cguuuuagagcuagaaaua | UGGCCguuuuagagcuag | GCC (TGG) (SEQ ID |
| | gcaaguuaaaauaaggcua | aaauagcaaguuaaaaua | NO: 12) |
| | guccguuaucaacuugaaa | aggcuaguccguuaucaa | GCTACTCTCTCTTTCTG |
| | aaguggcaccgagucggug | cuugaaaaaguggcaccg | GCC (SEQ ID NO: |
| | CUUUU (SEQ ID NO: | agucggugcu*U*U*U | 13) |
| | 8) | (SEQ ID NO: 9) | |
| β2M-1 | GCUACUCUCUCUUUCUGGC | G*C*U*ACUCUCUCUUUC | |
| sgRNA | C (SEQ ID NO: 10) | UGGCC (SEQ ID NO: | |
| spacer | | 11) | |
| β2M-4 | CAGUAAGUCAACUUCAAUG | C*A*G*UAAGUCAACUUC | CAGTAAGTCAACTTCAA |
| sgRNA | Uguuuuagagcuagaaaua | AAUGUguuuuagagcuag | TGT(CGG) (SEQ ID |
| | gcaaguuaaaauaaggcua | aaauagcaaguuaaaaua | NO: 18) |
| | guccguuaucaacuugaaa | aggcuaguccguuaucaa | CAGTAAGTCAACTTCAA |
| | aaguggcaccgagucggug | cuugaaaaaguggcaccg | TGT (SEQ ID NO: |
| | CUUUU | agucggugcu*U*U*U | 19) |
| | (SEQ ID NO: 14) | (SEQ ID NO: 15) | |
| β2M-4 | CAGUAAGUCAACUUCAAUG | C*A*G*UAAGUCAACUUC | |
| sgRNA | U | AAUGU | |
| spacer | (SEQ ID NO: 16) | (SEQ ID NO: 17) | |
| CD83 | GUAGGGAACCUGCGGAUCC | G*U*A*GGGAACCUGCGG | GTAGGGAACCTGCGGAT |
| exon2_3 | CAGGGUUUUAGAGCUAGAA | AUCCCAGGGUUUUAGAGC | CCCAGG (GGG) |
| sgRNA | AUAGCAAGUUAAAAUAAGG | UAGAAAUAGCAAGUUAAA | (SEQ ID NO: 24) |
| | CUAGUCCGUUAUCAACUUG | AUAAGGCUAGUCCGUUAU | GTAGGGAACCTGCGGAT |
| | AAAAAGUGGCACCGAGUCG | CAACUUGAAAAAGUGGCA | CCCAGG |
| | GUGCUUUU | CCGAGUCGGUGCU*U*U* | (SEQ ID NO: 25) |
| | (SEQ ID NO: 20) | U | |
| | | (SEQ ID NO: 21) | |
| CD83 | GUAGGGAACCUGCGGAUCC | G*U*A*GGGAACCUGCGG | |
| exon2_3 | CAGG | AUCCCAGG | |
| sgRNA | (SEQ ID NO: 22) | (SEQ ID NO: 23) | |
| spacer | | | |
| CD83_exon2_ | AGGUUCCCUACACGGUCUC | A*G*G*UUCCCUACACGG | AGGTTCCCTACACGGTC |
| G2 sgRNA | CGUUUUAGAGCUAGAAAUA | UCUCCGUUUUAGAGCUAG | TCC (TGG) (SEQ ID |
| (+) | GCAAGUUAAAAUAAGGCUA | AAAUAGCAAGUUAAAAUA | NO: 100) |
| | GUCCGUUAUCAACUUGAAA | AGGCUAGUCCGUUAUCAA | AGGTTCCCTACACGGTC |
| | AAGUGGCACCGAGUCGGUG | CUUGAAAAAGUGGCACCG | TCC |
| | CU (SEQ ID NO: 96) | AGUCGGU*G*C*U (SEQ | (SEQ ID NO: 101) |
| | | ID NO: 97) | |
| CD83_exon2_ | AGGUUCCCUACACGGUCUC | A*G*G*UUCCCUACACGG | |
| G2 spacer | C (SEQ ID NO: 98) | UCUCC (SEQ ID NO: | |
| | | 99) | |
| TGFBRII | CCCCUACCAUGACUUUAUU | C*C*C*CUACCAUGACUU | CCCCTACCATGACTTTA |
| sgRNA | Cguuuuagagcuagaaaua | UAUUCguuuuagagcuag | TTC (TGG) (SEQ ID |
| (EX5_T1) | gcaaguuaaaauaaggcua | aaauagcaaguuaaaaua | NO: 30) |
| | guccguuaucaacuugaaa | aggcuaguccguuaucaa | CCCCTACCATGACTTTA |
| | aaguggcaccgagucggug | cuugaaaaaguggcaccg | TTC (SEQ ID NO: |
| | CUUUU | agucggugcu*U*U*U | 31) |
| | (SEQ ID NO: 26) | (SEQ ID NO: 27) | |
| TGFBRII | CCCCUACCAUGACUUUAUU | C*C*C*CUACCAUGACUU | |
| sgRNA | C (SEQ ID NO: 28) | UAUUC (SEQ ID NO: | |
| (EX5_T1) | | 29) | |
| spacer | | | |
| REG1-Z10 | ACGACGCGUGGGUGGCAAG | A*C*G*ACGCGUGGGUGG | ACGACGCGTGGGTGGCA |
| sgRNA | Cguuuuagagcuagaaaua | CAAGCguuuuagagcuag | AGC(GGG) (SEQ ID |
| (EX4_T7) | gcaaguuaaaauaaggcua | aaauagcaaguuaaaaua | NO: 36) |
| | guccguuaucaacuugaaa | aggcuaguccguuaucaa | ACGACGCGTGGGTGGCA |
| | aaguggcaccgagucggug | cuugaaaaaguggcaccg | AGC |
| | cUUUU (SEQ ID NO: | agucggugcu*U*U*U | (SEQ ID NO: 37) |
| | 32) | (SEQ ID NO: 33) | |
| REG1-Z10 | ACGACGCGUGGGUGGCAAG | A*C*G*ACGCGUGGGUGG | |
| sgRNA | C (SEQ ID NO: 34) | CAAGC (SEQ ID NO: | |
| (EX4_T7) | | 35) | |
| spacer | | | |

Exemplary sgRNA Formula
nnnnnnnnnnnnnnnnnnnnguuuuagagcuagaaauagcaaguuaaaauaaggcuaguccguuaucaacuu
gaaaaaguggcaccgagucggugcuuuu (SEQ ID NO: 38)
nnnnnnnnnnnnnnnnnnnnguuuuagagcuagaaauagcaaguuaaaauaaggcuaguccguuaucaacuu
gaaaaaguggcaccgagucggugc (SEQ ID NO: 39)
n₍₁₇₋₃₀₎guuuuagagcuagaaauagcaaguuaaaauaaggcuaguccguuaucaacuug
aaaaaguggcaccgagucggugcu₍₁₋₈₎ (SEQ ID NO: 40)
*2′-O-methyl phosphorothioate residue

TABLE 18
Exemplary Nucleotide Sequences in Disrupted TRAC Gene

| | Sequence (Deletions indicated by dashes (-); | |
|---|---|---|
| Description | insertions indicated by bold) | SEQ ID NO: |
| TRAC gene | AA---------------------GAGCAACAAATCTGACT | 41 |
| edit | | |
| TRAC gene | AAGAGCAACAGTGCTGT-GCCTGGAGCAACAAATCTGACT | 42 |
| edit | | |
| TRAC gene | AAGAGCAACAGTG-------CTGGAGCAACAAATCTGACT | 43 |
| edit | | |
| TRAC gene | AAGAGCAACAGT------GCCTGGAGCAACAAATCTGACT | 44 |
| edit | | |
| TRAC gene | AAGAGCAACAGTG---------------------CTGACT | 45 |
| edit | | |
| TRAC gene | AAGAGCAACAGTGCTGTGGGCCTGGAGCAACAAATCTGACT | 46 |
| edit | | |
| TRAC gene | AAGAGCAACAGTGC--TGGCCTGGAGCAACAAATCTGACT | 47 |
| edit | | |
| TRAC gene | AAGAGCAACAGTGCTGTGTGCCTGGAGCAACAAATCTGACT | 48 |
| edit | | |

TABLE 19
Exemplary Nucleotide Sequences in Disrupted β2M Gene

| | Sequence (Deletions indicated by dashes (-); | SEQ ID |
|---|---|---|
| Description | insertions indicated by bold) | NO: |
| β2M gene-edit | CGTGGCCTTAGCTGTGCTCGCGCTACTCTCTCTTTCT- | 49 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 50 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 51 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTGGAGGC | 52 |
| | TATCCAGCGTGAGTCTCTCCTACCCTCCCGCT | |
| β2M gene-edit | CGTGGCCTTAGCTGTGCTCGC------------------------- | 53 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGAGGCTA | 54 |
| | TCCAGCGTGAGTCTCTCCTACCCTCCCGCT | |

TABLE 20
Exemplary Nucleotide Sequences in Disrupted
TGFBRII Gene

| | | SEQ |
|---|---|---|
| | | ID |
| Description | Gene Edited Sequence^{b} | NO: |
| TGFBRII Gene-edit | CATGA-------CTGGAAGA | 55 |
| TGFBRII Gene-edit | CATGAC----TTCTGGAAGA | 56 |
| TGFBRII Gene-edit | CATGACT---TTCTGGAAGA | 57 |
| TGFBRII Gene-edit | CATGACTTTATTTCTGGAAGA | 58 |
| TGFBRII Gene-edit | CATGACTTTAATTCTGGAAGA | 59 |
| TGFBRII Gene-edit | CA-----------TGGAAGA | |
| TGFBRII Gene-edit | CATGACTT--TTCTGGAAGA | 60 |
| TGFBRII Gene-edit | CAT------------GAAGA | |
| TGFBRII Gene-edit | C------------------A | |
| TGFBRII Gene-edit | -------------------- | |
| TGFBRII Gene-edit | CATGA--------------- | |
| TGFBRII Gene-edit | CAT---------------GA | |
| TGFBRII Gene-edit | CA---------TCTGGAAGA | 61 |
| TGFBRII Gene-edit | CATGACTTT-TTCTGGAAGA | 62 |
| TGFBRII Gene-edit | CATGACTTTA-TCTGGAAGA | 63 |
| TGFBRII Gene-edit | CATGACTTT-------AAGA | 64 |
| TGFBRII Gene-edit | ----------TTCTGGAAGA | 65 |
| TGFBRII Gene-edit | CATGACTTTA--CTGGAAGA | 66 |

TABLE 21
Exemplary Nucleotide Sequences in Disrupted Reg1
Gene

| Description | Gene Edited Sequence^{b} | SEQ ID NO: |
|---|---|---|
| Reg1 Gene-edit | GTGGGTGGCAAAGCGGGTGGT | 67 |
| Reg1 Gene-edit | GT-----------GGGTGGT | |
| Reg1 Gene-edit | -----------GCGGGTGGT | |
| Reg1 Gene-edit | GTGGGTGGC-AGCGGGTGGT | 68 |
| Reg1 Gene-edit | ---------------GTGGT | |
| Reg1 Gene-edit | GTG--------------GGT | |
| Reg1 Gene-edit | ------------CGGGTGGT | |
| Reg1 Gene-edit | -------------------- | |
| Reg1 Gene-edit | GTGGGTGGC----------- | |
| Reg1 Gene-edit | GTGGGTGGCATAGCGGGTGGT | 69 |
| Reg1 Gene-edit | GTGGGTG------------- | |
| Reg1 Gene-edit | GTGG---------------- | |
| Reg1 Gene-edit | GTGGGTGG--AGCGGGTGGT | 70 |

TABLE 22
Exemplary Sequences of Anti-CD83 CAR Components

| | | SEQ ID |
|---|---|---|
| Name | Sequence | NO: |
| anti-CD83 | TGGYWWT | 71 |
| antibody | | |
| VH CDR1 | | |
| anti-CD83 | YIFSSGNTNYNPSIKS | 72 |
| antibody | | |
| VH CDR2 | | |
| anti-CD83 | AYGKLGFDY | 73 |
| antibody | | |
| VH CDR3 | | |
| anti-CD83 | TLSSQHSTYTIG | 74 |
| antibody | | |
| VL CDR1 | | |
| anti-CD83 | VNSDGSHSKGD | 75 |
| antibody | | |
| VL CDR2 | | |
| anti-CD83 | GSSDSSGYV | 76 |
| antibody | | |
| VL CDR3 | | |
| Anti-CD83 | QVQLKESGPGLVKPSQSLSLTCSVTGFSITTGGYWWTWIRQFPGQKLEWMG | 77 |
| VH | YIFSSGNTNYNPSIKSRISITRDTSKNQFFLQLNSVTTEGDTARYYCARAY | |
| | GKLGFDYWGQGTLVTVS | |
| Anti-CD83 | QPVLTQSPSASASLGNSVKITCTLSSQHSTYTIGWYQQHPDKAPKYVMYVN | 78 |
| VL | SDGSHSKGDGIPDRFSGSSSGAHRYLSISNIQPEDEADYFCGSSDSSGYVF | |
| | GSGTQLTVL | |
| Anti-CD83 | QVQLKESGPGLVKPSQSLSLTCSVTGFSITTGGYWWTWIRQFPGQKLEWMG | 79 |
| scFv | YIFSSGNTNYNPSIKSRISITRDTSKNQFFLQLNSVTTEGDTARYYCARAY | |
| | GKLGFDYWGQGTLVTVSSGGGGSGGGGSGGGGSQPVLTQSPSASASLGNSV | |
| | KITCTLSSQHSTYTIGWYQQHPDKAPKYVMYVNSDGSHSKGDGIPDRFSGS | |
| | SSGAHRYLSISNIQPEDEADYFCGSSDSSGYVFGSGTQLTVL | |
| Anti-CD83 | MALPVTALLLPLALLLHAARPQVQLKESGPGLVKPSQSLSLTCSVTGFSIT | 80 (with |
| CAR | TGGYWWTWIRQFPGQKLEWMGYIFSSGNTNYNPSIKSRISITRDTSKNQFF | signal |
| | LQLNSVTTEGDTARYYCARAYGKLGFDYWGQGTLVTVSSGGGGSGGGGSGG | peptide) |
| | GGSQPVLTQSPSASASLGNSVKITCTLSSQHSTYTIGWYQQHPDKAPKYVM | 81 (no |
| | YVNSDGSHSKGDGIPDRFSGSSSGAHRYLSISNIQPEDEADYFCGSSDSSG | signal |
| | YVFGSGTQLTVLRAAATTTPAPRPPTPAPTIASQPLSLRPEACRPAAGGAV | peptide) |
| | HTRGLDFACDIYIWAPLAGTCGVLLLSLVITLYCKRGRKKLLYIFKQPFMR | |
| | PVQTTQEEDGCSCRFPEEEEGGCELRVKFSRSADAPAYKOGQNQLYNELNL | |
| | GRREEYDVLDKRRGRDPEMGGKPRRKNPQEGLYNELQKDKMAEAYSEIGMK | |
| | GERRRGKGHDGLYQGLSTATKDTYDALHMQALPPR | |
| Signal | MALPVTALLLPLALLLHAARP | 82 |
| Peptide 1 | | |
| Signal | MLLLVTSLLLCELPHPAFLLIP | 83 |
| Peptide 2 | | |
| 4-1BB CoS | KRGRKKLLYIFKQPFMRPVQTTQEEDGCSCRFPEEEEGGCEL | 84 |
| CD28 CoS | SKRSRLLHSDYMNMTPRRPGPTRKHYQPYAPPRDFAAYRS | 85 |
| CD3ζ | RVKFSRSADAPAYQQGQNQLYNELNLGRREEYDVLDKRRGRDPEMGGKPRR | 86 |
| signaling | KNPQEGLYNELQKDKMAEAYSEIGMKGERRRGKGHDGLYQGLSTATKDTYD | |
| | ALHMQALPPR | |
| CD8a | TTTPAPRPPTPAPTIASQPLSLRPEACRPAAGGAVHTRGLDFACDIYIWAP | 87 |
| TM/Hinge | LAGTCGVLLLSLVITLYC | |

CA

TABLE 23
Exemplary AAV Donor Template Sequences

| | | SEQ ID |
|---|---|---|
| Name | Sequence | NO: |
| Left ITR | TTGGCCACTCCCTCTCTGCGCGCTCGCTCGCTCACTGAGGCCGGGCGACCAAA | 88 |
| (5′ ITR) | GGTCGCCCGACGCCCGGGCTTTGCCCGGGCGGCCTCAGTGAGCGAGCGAGCGC | |
| | GCAGAGAGGGAGTGGCCAACTCCATCACTAGGGGTTCCT | |
| Left ITR | CCTGCAGGCAGCTGCGCGCTCGCTCGCTCACTGAGGCCGCCCGGGCGTCGGGC | 89 |
| (5′ ITR) | GACCTTTGGTCGCCCGGCCTCAGTGAGCGAGCGAGCGCGCAGAGAGGGAGTGG | |
| (alternate) | CCAACTCCATCACTAGGGGTTCCT | |
| Right ITR | AGGAACCCCTAGTGATGGAGTTGGCCACTCCCTCTCTGCGCGCTCGCTCGCTC | 90 |
| (3′ ITR) | ACTGAGGCCGCCCGGGCAAAGCCCGGGCGTCGGGCGACCTTTGGTCGCCCGGC | |
| | CTCAGTGAGCGAGCGAGCGCGCAGAGAGGGAGTGGCCAA | |
| Right ITR | AGGAACCCCTAGTGATGGAGTTGGCCACTCCCTCTCTGCGCGCTCGCTCGCTC | 91 |
| (3′ ITR) | ACTGAGGCCGGGCGACCAAAGGTCGCCCGACGCCCGGGCTTTGCCCGGGCGGC | |
| (alternate) | CTCAGTGAGCGAGCGAGCGCGCAGCTGCCTGCAGG | |
| TRAC- | GAGATGTAAGGAGCTGCTGTGACTTGCTCAAGGCCTTATATCGAGTAAACGGT | 92 |
| LHA | AGTGCTGGGGCTTAGACGCAGGTGTTCTGATTTATAGTTCAAAACCTCTATCA | |
| (800 bp) | ATGAGAGAGCAATCTCCTGGTAATGTGATAGATTTCCCAACTTAATGCCAACA | |
| | TACCATAAACCTCCCATTCTGCTAATGCCCAGCCTAAGTTGGGGAGACCACTC | |
| | CAGATTCCAAGATGTACAGTTTGCTTTGCTGGGCCTTTTTCCCATGCCTGCCT | |
| | TTACTCTGCCAGAGTTATATTGCTGGGGTTTTGAAGAAGATCCTATTAAATAA | |
| | AAGAATAAGCAGTATTATTAAGTAGCCCTGCATTTCAGGTTTCCTTGAGTGGC | |
| | AGGCCAGGCCTGGCCGTGAACGTTCACTGAAATCATGGCCTCTTGGCCAAGAT | |
| | TGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGA | |
| | GCCCCGCCCTTGTCCATCACTGGCATCTGGACTCCAGCCTGGGTTGGGGCAAA | |
| | GAGGGAAATGAGATCATGTCCTAACCCTGATCCTCTTGTCCCACAGATATCCA | |
| | GAACCCTGACCCTGCCGTGTACCAGCTGAGAGACTCTAAATCCAGTGACAAGT | |
| | CTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGA | |
| | CTTCA | |
| TRAC- | TGGAGCAACAAATCTGACTTTGCATGTGCAAACGCCTTCAACAACAGCATTAT | 93 |
| RHA | TCCAGAAGACACCTTCTTCCCCAGCCCAGGTAAGGGCAGCTTTGGTGCCTTCG | |
| (800 bp) | CAGGCTGTTTCCTTGCTTCAGGAATGGCCAGGTTCTGCCCAGAGCTCTGGTCA | |
| | ATGATGTCTAAAACTCCTCTGATTGGTGGTCTCGGCCTTATCCATTGCCACCA | |
| | AAACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGCCC | |
| | AGCCTCAGTCTCTCCAACTGAGTTCCTGCCTGCCTGCCTTTGCTCAGACTGTT | |
| | TGCCCCTTACTGCTCTTCTAGGCCTCATTCTAAGCCCCTTCTCCAAGTTGCCT | |
| | CTCCTTATTTCTCCCTGTCTGCCAAAAAATCTTTCCCAGCTCACTAAGTCAGT | |
| | CTCACGCAGTCACTCATTAACCCACCAATCACTGATTGTGCCGGCACATGAAT | |
| | GCACCAGGTGTTGAAGTGGAGGAATTAAAAAGTCAGATGAGGGGTGTGCCCAG | |
| | AGGAAGCACCATTCTAGTTGGGGGAGCCCATCTGTCAGCTGGGAAAAGTCCAA | |
| | ATAACTTCAGATTGGAATGTGTTTTAACTCAGGGTTGAGAAAACAGCTACCTT | |
| | CAGGACAAAAGTCAGGGAAGGGCTCTCTGAAGAAATGCTACTTGAAGATACCA | |
| | GCCCTACCAAGGGCAGGGAGAGGACCCTATAGAGGCCTGGGACAGGAGCTCAA | |
| | TGAGAAAGG | |
| EF1α | GGCTCCGGTGCCCGTCAGTGGGCAGAGCGCACATCGCCCACAGTCCCCGAGAA | 94 |
| | GTTGGGGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGTGGG | |
| | GGAGAACCGTATATAAGTGCAGTAGTCGCCGTGAACGTTCTTTTTCGCAACGG | |
| | GTTTGCCGCCAGAACACAGGTAAGTGCCGTGTGTGGTTCCCGCGGGCCTGGCC | |
| | TCTTTACGGGTTATGGCCCTTGCGTGCCTTGAATTACTTCCACTGGCTGCAGT | |
| | ACGTGATTCTTGATCCCGAGCTTCGGGTTGGAAGTGGGTGGGAGAGTTCGAGG | |
| | CCTTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGCTG | |
| | CTTTCGATAAGTCTCTAGCCATTTAAAATTTTTGATGACCTGCTGCGACGCTT | |
| | TTTTTCTGGCAAGATAGTCTTGTAAATGCGGGCCAAGATCTGCACACTGGTAT | |
| | TTCGGTTTTTGGGGCCGCGGGCGGCGACGGGGCCCGTGCGTCCCAGCGCACAT | |
| | GTTCGGCGAGGCGGGGCCTGCGAGCGCGGCCACCGAGAATCGGACGGGGGTAG | |
| | TCTCAAGCTGGCCGGCCTGCTCTGGTGCCTGGCCTCGCGCCGCCGTGTATCGC | |
| | CCCGCCCTGGGCGGCAAGGCTGGCCCGGTCGGCACCAGTTGCGTGAGCGGAAA | |
| | GATGGCCGCTTCCCGGCCCTGCTGCAGGGAGCTCAAAATGGAGGACGCGGCGC | |
| | TCGGGAGAGCGGGCGGGTGAGTCACCCACACAAAGGAAAAGGGCCTTTCCGTC | |
| | CTCAGCCGTCGCTTCATGTGACTCCACGGAGTACCGGGCGCCGTCCAGGCACC | |
| | TCGATTAGTTCTCGAGCTTTTGGAGTACGTCGTCTTTAGGTTGGGGGGAGGGG | |
| | TTTTATGCGATGGAGTTTCCCCACACTGAGTGGGTGGAGACTGAAGTTAGGCC | |
| | AGCTTGGCACTTGATGTAATTCTCCTTGGAATTTGCCCTTTTTGAGTTTGGAT | |
| | CTTGGTTCATTCTCAAGCCTCAGACAGTGGTTCAAAGTTTTTTTCTTCCATTT | |
| | CAGGTGTCGTGA | |
| LHA- | GAGATGTAAGGAGCTGCTGTGACTTGCTCAAGGCCTTATATCGAGTAAACGGT | 95 |
| CD83 | AGTGCTGGGGCTTAGACGCAGGTGTTCTGATTTATAGTTCAAAACCTCTATCA | |
| CAR- | ATGAGAGAGCAATCTCCTGGTAATGTGATAGATTTCCCAACTTAATGCCAACA | |
| RHA | TACCATAAACCTCCCATTCTGCTAATGCCCAGCCTAAGTTGGGGAGACCACTC | |
| | CAGATTCCAAGATGTACAGTTTGCTTTGCTGGGCCTTTTTCCCATGCCTGCCT | |
| | TTACTCTGCCAGAGTTATATTGCTGGGGTTTTGAAGAAGATCCTATTAAATAA | |
| | AAGAATAAGCAGTATTATTAAGTAGCCCTGCATTTCAGGTTTCCTTGAGTGGC | |
| | AGGCCAGGCCTGGCCGTGAACGTTCACTGAAATCATGGCCTCTTGGCCAAGAT | |
| | TGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGA | |
| | GCCCCGCCCTTGTCCATCACTGGCATCTGGACTCCAGCCTGGGTTGGGGCAAA | |
| | GAGGGAAATGAGATCATGTCCTAACCCTGATCCTCTTGTCCCACAGATATCCA | |
| | GAACCCTGACCCTGCCGTGTACCAGCTGAGAGACTCTAAATCCAGTGACAAGT | |
| | CTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGA | |
| | CTTCAGGCTCCGGTGCCCGTCAGTGGGCAGAGCGCACATCGCCCACAGTCCCC | |
| | GAGAAGTTGGGGGGAGGGGTCGGCAATTGAACCGGTGCCTAGAGAAGGTGGCG | |
| | CGGGGTAAACTGGGAAAGTGATGTCGTGTACTGGCTCCGCCTTTTTCCCGAGG | |
| | GTGGGGGAGAACCGTATATAAGTGCAGTAGTCGCCGTGAACGTTCTTTTTCGC | |
| | AACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGCT | |
| | GCAGTACGTGATTCTTGATCCCGAGCTTCGGGTTGGAAGTGGGTGGGAGAGTT | |
| | CGAGGCCTTGCGCTTAAGGAGCCCCTTCGCCTCGTGCTTGAGTTGAGGCCTGG | |
| | CCTGGGCGCTGGGGCCGCCGCGTGCGAATCTGGTGGCACCTTCGCGCCTGTCT | |
| | CGCTGCTTTCGATAAGTCTCTAGCCATTTAAAATTTTTGATGACCTGCTGCGA | |
| | CGCTTTTTTTCTGGCAAGATAGTCTTGTAAATGCGGGCCAAGATCTGCACACT | |
| | GGTATTTCGGTTTTTGGGGCCGCGGGCGGCGACGGGGCCCGTGCGTCCCAGCG | |
| | CACATGTTCGGCGAGGCGGGGCCTGCGAGCGCGGCCACCGAGAATCGGACGGG | |
| | GGTAGTCTCAAGCTGGCCGGCCTGCTCTGGTGCCTGGCCTCGCGCCGCCGTGT | |
| | ATCGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGACGC | |
| | GGCGCTCGGGAGAGCGGGCGGGTGAGTCACCCACACAAAGGAAAAGGGCCTTT | |
| | CCGTCCTCAGCCGTCGCTTCATGTGACTCCACGGAGTACCGGGCGCCGTCCAG | |
| | GCACCTCGATTAGTTCTCGAGCTTTTGGAGTACGTCGTCTTTAGGTTGGGGGG | |
| | AGGGGTTTTATGCGATGGAGTTTCCCCACACTGAGTGGGTGGAGACTGAAGTT | |
| | AGGCCAGCTTGGCACTTGATGTAATTCTCCTTGGAATTTGCCCTTTTTGAGTT | |
| | TGGATCTTGGTTCATTCTCAAGCCTCAGACAGTGGTTCAAAGTTTTTTTCTTC | |
| | CATTTCAGGTGTCGTGACCACCATGGCGCTTCCGGTGACAGCACTGCTCCTCC | |
| | CCTTGGCGCTGTTGCTCCACGCAGCAAGGCCGCAGGTTCAGCTGAAAGAATCT | |
| | GGCCCCGGCCTGGTTAAACCGAGTCAGAGTTTGAGCTTGACTTGTTCTGTGAC | |
| | TGGATTCTCAATAACAACCGGTGGATACTGGTGGACCTGGATACGACAATTTC | |
| | CGGGCCAAAAGCTCGAGTGGATGGGCTATATCTTTAGTAGCGGCAACACTAAC | |
| | TATAATCCTTCAATCAAGAGCAGGATAAGTATCACCCGAGACACCAGTAAGAA | |
| | CCAATTCTTTTTGCAGCTTAACAGCGTTACGACTGAAGGTGACACCGCAAGGT | |
| | ATTACTGCGCGAGGGCCTACGGAAAACTTGGTTTTGATTATTGGGGTCAAGGG | |
| | ACACTGGTAACGGTTTCTAGTGGGGGAGGTGGTAGTGGCGGTGGAGGTTCCGG | |
| | GGGTGGAGGATCCCAACCCGTCCTTACCCAGAGTCCTTCTGCGTCCGCTAGTC | |
| | TCGGAAATAGTGTTAAGATTACGTGCACTTTGAGTAGCCAACATTCCACTTAT | |
| | ACCATCGGCTGGTATCAGCAACACCCAGATAAAGCCCCGAAATATGTGATGTA | |
| | TGTCAATTCCGACGGTTCTCATTCAAAAGGCGACGGGATCCCCGACAGATTCA | |
| | GCGGTAGTTCATCTGGAGCTCACCGCTATCTGTCAATATCCAATATTCAGCCG | |
| | GAAGATGAAGCTGACTATTTTTGCGGGAGCAGTGATAGTTCCGGTTATGTCTT | |
| | CGGCAGTGGTACACAGCTTACCGTACTCAGAGCAGCCGCTACAACAACTCCTG | |
| | CACCGCGACCTCCAACGCCTGCCCCTACCATTGCGTCACAACCACTTAGCCTT | |
| | AGGCCAGAAGCGTGCCGACCTGCAGCAGGCGGGGCCGTTCATACTAGGGGTCT | |
| | TGACTTCGCGTGCGATATCTACATATGGGCACCTCTGGCGGGAACCTGTGGTG | |
| | TGTTGCTTCTTAGTCTGGTAATTACACTCTACTGTAAACGGGGCAGAAAGAAA | |
| | CTCCTGTATATATTCAAACAACCATTTATGAGACCAGTACAAACTACTCAAGA | |
| | GGAAGATGGCTGTAGCTGCCGATTTCCAGAAGAAGAAGAAGGAGGATGTGAAC | |
| | TGCGCGTGAAGTTTTCACGATCTGCAGATGCCCCGGCATATAAACAAGGTCAG | |
| | AACCAGTTGTACAATGAATTGAACCTTGGTAGAAGGGAGGAGTATGACGTCCT | |
| | CGACAAGAGAAGAGGGCGCGACCCGGAGATGGGCGGTAAGCCAAGGAGAAAAA | |
| | ATCCGCAAGAGGGGCTTTATAATGAACTCCAGAAGGATAAGATGGCTGAGGCT | |
| | TACAGTGAGATTGGTATGAAAGGAGAAAGAAGACGCGGAAAGGGACATGATGG | |
| | GTTGTACCAAGGGCTCAGCACTGCTACCAAGGACACTTATGACGCGCTCCATA | |
| | TGCAAGCCCTTCCCCCTAGATAATAATAAAATCGCTATCCATCGAAGATGGAT | |
| | GTGTGTTGGTTTTTTGTGTGTGGAGCAACAAATCTGACTTTGCATGTGCAAAC | |
| | GCCTTCAACAACAGCATTATTCCAGAAGACACCTTCTTCCCCAGCCCAGGTAA | |
| | GGGCAGCTTTGGTGCCTTCGCAGGCTGTTTCCTTGCTTCAGGAATGGCCAGGT | |
| | TCTGCCCAGAGCTCTGGTCAATGATGTCTAAAACTCCTCTGATTGGTGGTCTC | |
| | GGCCTTATCCATTGCCACCAAAACCCTCTTTTTACTAAGAAACAGTGAGCCTT | |
| | GTTCTGGCAGTCCAGAGAATGACACGGGAAAAAAGCAGATGAAGAGAAGGTGG | |
| | CAGGAGAGGGCACGTGGCCCAGCCTCAGTCTCTCCAACTGAGTTCCTGCCTGC | |
| | CTGCCTTTGCTCAGACTGTTTGCCCCTTACTGCTCTTCTAGGCCTCATTCTAA | |
| | GCCCCTTCTCCAAGTTGCCTCTCCTTATTTCTCCCTGTCTGCCAAAAAATCTT | |
| | TCCCAGCTCACTAAGTCAGTCTCACGCAGTCACTCATTAACCCACCAATCACT | |
| | GATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATCT | |
| | GTCAGCTGGGAAAAGTCCAAATAACTTCAGATTGGAATGTGTTTTAACTCAGG | |
| | GTTGAGAAAACAGCTACCTTCAGGACAAAAGTCAGGGAAGGGCTCTCTGAAGA | |
| | AATGCTACTTGAAGATACCAGCCCTACCAAGGGCAGGGAGAGGACCCTATAGA | |
| | GGCCTGGGACAGGAGCTCAATGAGAAAGG | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A population of genetically engineered T cells, comprising:
(i) a nucleic acid encoding a chimeric antigen receptor (CAR) that binds CD83 (anti-CD83 CAR); and
(ii) a disrupted Regnase-1 (Reg1) gene; a disrupted Transforming Growth Factor Beta Receptor II (TGFBRII) gene, or a combination thereof.

2. The population of genetically engineered T cells of claim 1, which comprises both the disrupted Reg1 gene and the disrupted TGFBRII gene.

3. The population of genetically engineered T cells Of of claim 1, which further comprises (iii) a disrupted CD83 gene.

4. The population of genetically engineered T cells of claim 3, which further comprises (iv) a disrupted T cell receptor alpha chain constant region (TRAC) gene, a disrupted beta-2-microglobulin (β2M) gene, or a combination thereof.

5. The population of genetically engineered T cells of claim 1, which comprises:
(i) the nucleic acid encoding the anti-CD83 CAR;
(ii) the disrupted Reg1 gene and the disrupted TGFBRII gene;
(iii) the disrupted CD83 gene; and
(iv) the disrupted TRAC gene and the disrupted β2M gene.

6. The population of genetically engineered T cells of claim 1, wherein the disrupted Reg1 gene is genetically edited in exon 4; and/or wherein the disrupted TGFBRII gene is genetically edited in exon 5.

7. (canceled)

8. The population of genetically engineered T cells of claim 3, wherein the disrupted CD83 gene is genetically edited in exon 2 or exon 3.

9. The population of genetically engineered T cells of claim 4, wherein the disrupted CD83 gene, the disrupted Reg1 gene, the disrupted TGFBRII gene, the disrupted TRAC gene, and/or the disrupted β2M gene are genetically edited by a CRISPR/Cas-mediated gene editing system.

10. The population of genetically engineered T cells of claim 9, wherein the CRISPR/Cas-mediated gene editing comprises:
(a) a guide RNA (gRNA) targeting a site in the Reg1 gene that comprises the nucleotide sequence of SEQ ID NO:36 or 37;
(b) a gRNA targeting a site in the TGFBRII gene that comprises the nucleotide sequence of SEQ ID NO: 30 or 31;
(c) a gRNA targeting a site in the CD83 gene that comprises the nucleotide sequence of SEQ ID NO: 24 or 25, or SEQ ID NO: 100 or 101;
(d) a guide RNA (gRNA) targeting a site in the TRAC gene that comprises the nucleotide sequence of SEQ ID NO: 6 or 7; and/or
(e) a gRNA targeting a site in the β2M gene that comprises the nucleotide sequence of SEQ ID NO: 12 or 13, or SEQ ID NO: 18 or 19.

11. (canceled)

12. The population of genetically engineered T cells of claim 10, wherein the CRISPR/Cas-mediated gene editing comprises:
(a) the gRNA targeting the Reg1 gene comprises a spacer, which comprises the nucleotide sequence of SEQ ID NO: 34;
(b) the gRNA targeting the TGFBRII gene comprises a spacer, which comprises the nucleotide sequence of SEQ ID NO: 28;
(c) the gRNA targeting the CD83 gene comprises a spacer, which comprises the nucleotide sequence of SEQ ID NO: 22 or 98;
(d) the gRNA targeting the TRAC gene comprises a spacer, which comprises the nucleotide sequence of SEQ ID NO: 4; and/or
(e) the gRNA targeting the β2M gene comprises a spacer, which comprises the nucleotide sequence of SEQ ID NO:10 or 16.

13. The population of genetically engineered T cells of claim 1, wherein the nucleic acid encoding the anti-CD83 CAR is inserted in the genome of the T cells.

14. The population of genetically engineered T cells of claim 13, wherein the nucleic acid encoding the anti-CD83 CAR is inserted in the disrupted CD83 gene, the disrupted Reg1 gene, the disrupted TGFBRII gene, the disrupted TRAC gene, or the disrupted β2M.

15. The population of genetically engineered T cells of claim 14, wherein the nucleic acid encoding the anti-CD83 CAR is inserted in the disrupted TRAC gene.

16. The population of genetically engineered T cells of claim 15, wherein the nucleic acid encoding the anti-CD83 CAR replaces the deleted fragment comprising SEQ ID NO:7 in the TRAC gene.

17. The population of genetically engineered T cells of claim 1, wherein the CAR comprises an extracellular antigen binding domain specific to CD83, a co-stimulatory signaling domain of 4-1BB or CD28, and a cytoplasmic signaling domain of CD3.

18. The population of genetically engineered T cells of claim 17, wherein the extracellular antigen binding domain is a single chain variable fragment (scFv) that binds CD83 (anti-CD83 scFv), and wherein the anti-CD83 scFv comprises a heavy chain variable region (V_{H}) and a light chain variable region (V_{L}).

19. The population of genetically engineered T cells of claim 18, wherein the V_{H} comprises heavy chain complementary determining region (CDR) 1, CDR2, and CDR3 set forth as SEQ ID NOs:71, 72, and 73, respectively; and/or wherein the V_{L} comprises light chain complementary determining region (CDR) 1, CDR2, and CDR3 set forth as SEQ ID NOs:74, 75 and 76, respectively.

20. The population of genetically engineered T cells of claim 19, wherein the V_{H} comprises the amino acid sequence of SEQ ID NO: 77, and/or wherein the V_{L} comprises the amino acid sequence of SEQ ID NO: 78.

21. The population of genetically engineered T cells of claim 20, wherein the anti-CD83 scFv comprises the amino acid sequence of SEQ ID NO:79.

22. The population of genetically engineered T cells of claim 21, wherein the CAR that binds CD83 comprises the amino acid sequence of SEQ ID NO: 80 or 81.

23. The population of genetically engineered T cells of claim 1, wherein the genetically engineered T cells are derived from primary T cells of one or more human donors.

24. The population of genetically engineered T cells of claim 1, wherein the T cells further express a chimeric antigen receptor (CAR) that binds a tumor antigen, which optionally is CD19, BCMA, or CD70.

25. A method for preparing a population of genetically engineered T cells, the method comprising:
(a) providing a plurality of cells, which are T cells or precursor cells thereof;
(b) delivering to the T cells a nucleic acid encoding a chimeric antigen receptor (CAR) that binds CD83 (“anti-CD83 CAR”);
(c) genetically editing a Reg1 gene, a TGFBRII gene, or a combination thereof;
thereby producing a population of genetically engineered T cells expressing the anti-CD83 CAR and having a Reg1 gene, a disrupted, TGFBRII gene or a combination thereof.

26-50. (canceled)

51. A population of genetically engineered T cells, which is prepared by a method of claim 25.

52. A method for eliminating undesired cells in a subject, the method comprising administering to a subject in need thereof a population of genetically engineered T cells set forth in claim 1.

53-66. (canceled)

67. The population of genetically engineered T cells of claim 24, wherein the CAR binds a tumor antigen.

68. The population of genetically engineered T cells of claim 67, wherein the tumor antigen is CD19, BCMA, or CD70.


SEQUENCE LISTING (101 sequences)

SEQ ID NO: 1 - protein, 1368 aa, synthetic construct [sequence omitted]

SEQ ID NO: 2 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 3 - RNA, 100 nt, synthetic construct; identical to SEQ ID NO: 2
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 4 - RNA, 20 nt, synthetic construct
  source 1..20: mol_type = other RNA
agagcaacagtgctgtggcc

SEQ ID NO: 5 - RNA, 20 nt, synthetic construct
  source 1..20: mol_type = other RNA
  misc_feature 1..4: note = modified with 2'-O-methyl phosphorothioate
agagcaacagtgctgtggcc

SEQ ID NO: 6 - DNA, 23 bp, synthetic construct
  source 1..23: mol_type = other DNA
agagcaacagtgctgtggcctgg

SEQ ID NO: 7 - DNA, 20 bp, synthetic construct
  source 1..20: mol_type = other DNA
agagcaacagtgctgtggcc

SEQ ID NO: 8 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 9 - RNA, 100 nt, synthetic construct; identical to SEQ ID NO: 8
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 10 - RNA, 20 nt, synthetic construct
  source 1..20: mol_type = other RNA
gctactctctctttctggcc

SEQ ID NO: 11 - RNA, 20 nt, synthetic construct
  source 1..20: mol_type = other RNA
  misc_feature 1..4: note = modified with 2'-O-methyl phosphorothioate
gctactctctctttctggcc

SEQ ID NO: 12 - DNA, 23 bp, synthetic construct
  source 1..23: mol_type = other DNA
gctactctctctttctggcctgg

SEQ ID NO: 13 - DNA, 20 bp, synthetic construct
  source 1..20: mol_type = other DNA
gctactctctctttctggcc

SEQ ID NO: 14 - RNA, 100 nt, synthetic construct
  source 1..100: mol_type = other RNA
cagtaagtcaacttcaatgtgttttagagctagaaatagcaagttaaaataaggctagtccgttatcaacttgaaaaagtggcaccgagtcggtgctttt

SEQ ID NO: 15 - RNA, 100 nt, synthetic construct; identical to SEQ ID NO: 14
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 16 - RNA, 20 nt, synthetic construct
  source 1..20: mol_type = other RNA
cagtaagtcaacttcaatgt

SEQ ID NO: 17 - RNA, 20 nt, synthetic construct
  source 1..20: mol_type = other RNA
  misc_feature 1..4: note = modified with 2'-O-methyl phosphorothioate
cagtaagtcaacttcaatgt

SEQ ID NO: 18 - DNA, 23 bp, synthetic construct
  source 1..23: mol_type = other DNA
cagtaagtcaacttcaatgtcgg

SEQ ID NO: 19 - DNA, 20 bp, synthetic construct
  source 1..20: mol_type = other DNA
cagtaagtcaacttcaatgt

SEQ ID NO: 20 - RNA, 103 nt, synthetic construct
  source 1..103: mol_type = other RNA
gtagggaacctgcggatcccagggttttagagctagaaatagcaagttaaaataaggctagtccgttatcaacttgaaaaagtggcaccgagtcggtgctttt

SEQ ID NO: 21 - RNA, 103 nt, synthetic construct; identical to SEQ ID NO: 20
  source 1..103: mol_type = other RNA
  misc_feature 1..4: note = modified with 2'-O-methyl phosphorothioate
  misc_feature 99..103: note = modified with 2'-O-methyl phosphorothioate

SEQ ID NO: 22 - RNA, 23 nt, synthetic construct
  source 1..23: mol_type = other RNA
gtagggaacctgcggatcccagg

SEQ ID NO: 23 - RNA, 23 nt, synthetic construct
  source 1..23: mol_type = other RNA
  misc_feature 1..4: note = modified with 2'-O-methyl phosphorothioate
gtagggaacctgcggatcccagg

SEQ ID NO: 24 - DNA, 26 bp, synthetic construct
  source 1..26: mol_type = other DNA
gtagggaacctgcggatcccaggggg

SEQ ID NO: 25 - DNA, 23 bp, synthetic construct
  source 1..23: mol_type = other DNA
gtagggaacctgcggatcccagg

SEQ ID NO: 26 - RNA, 100 nt, synthetic construct
  source 1..100: mol_type = other RNA
cccctaccatgactttattcgttttagagctagaaatagcaagttaaaataaggctagtccgttatcaacttgaaaaagtggcaccgagtcggtgctttt

SEQ ID NO: 27 - RNA, 100 nt, synthetic construct; identical to SEQ ID NO: 26
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 28 - RNA, 20 nt, synthetic construct
  source 1..20: mol_type = other RNA
cccctaccatgactttattc

SEQ ID NO: 29 - RNA, 20 nt, synthetic construct
  source 1..20: mol_type = other RNA
  misc_feature 1..4: note = modified with 2'-O-methyl phosphorothioate
cccctaccatgactttattc

SEQ ID NO: 30 - DNA, 23 bp, synthetic construct
  source 1..23: mol_type = other DNA
cccctaccatgactttattctgg

SEQ ID NO: 31 - DNA, 20 bp, synthetic construct
  source 1..20: mol_type = other DNA
cccctaccatgactttattc

SEQ ID NO: 32 - RNA, 100 nt, synthetic construct
  source 1..100: mol_type = other RNA
acgacgcgtgggtggcaagcgttttagagctagaaatagcaagttaaaataaggctagtccgttatcaacttgaaaaagtggcaccgagtcggtgctttt

SEQ ID NO: 33 - RNA, 100 nt, synthetic construct; identical to SEQ ID NO: 32
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 34 - RNA, 20 nt, synthetic construct
  source 1..20: mol_type = other RNA
acgacgcgtgggtggcaagc

SEQ ID NO: 35 - RNA, 20 nt, synthetic construct
  source 1..20: mol_type = other RNA
  misc_feature 1..4: note = modified with 2'-O-methyl phosphorothioate
acgacgcgtgggtggcaagc

SEQ ID NO: 36 - DNA, 23 bp, synthetic construct
  source 1..23: mol_type = other DNA
acgacgcgtgggtggcaagcggg

SEQ ID NO: 37 - DNA, 20 bp, synthetic construct
  source 1..20: mol_type = other DNA
acgacgcgtgggtggcaagc

SEQ ID NO: 38 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 39 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 40 - RNA, 111 nt, synthetic construct
  source 1..111: mol_type = other RNA
  misc_feature 18..30: note = n is a, c, g, u, or absent
  misc_feature 105..111: note = u or absent
nnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 41 - DNA, 19 bp, synthetic construct
  source 1..19: mol_type = other DNA
aagagcaacaaatctgact

SEQ ID NO: 42 - DNA, 39 bp, synthetic construct
  source 1..39: mol_type = other DNA
aagagcaacagtgctgtgcctggagcaacaaatctgact

SEQ ID NO: 43 - DNA, 33 bp, synthetic construct
  source 1..33: mol_type = other DNA
aagagcaacagtgctggagcaacaaatctgact

SEQ ID NO: 44 - DNA, 34 bp, synthetic construct
  source 1..34: mol_type = other DNA
aagagcaacagtgcctggagcaacaaatctgact

SEQ ID NO: 45 - DNA, 19 bp, synthetic construct
  source 1..19: mol_type = other DNA
aagagcaacagtgctgact

SEQ ID NO: 46 - DNA, 41 bp, synthetic construct
  source 1..41: mol_type = other DNA
aagagcaacagtgctgtgggcctggagcaacaaatctgact

SEQ ID NO: 47 - DNA, 38 bp, synthetic construct
  source 1..38: mol_type = other DNA
aagagcaacagtgctggcctggagcaacaaatctgact

SEQ ID NO: 48 - DNA, 41 bp, synthetic construct
  source 1..41: mol_type = other DNA
aagagcaacagtgctgtgtgcctggagcaacaaatctgact

SEQ ID NO: 49 - DNA, 79 bp, synthetic construct
  source 1..79: mol_type = other DNA
cgtggccttagctgtgctcgcgctactctctctttctgcctggaggctatccagcgtgagtctctcctaccctcccgct

SEQ ID NO: 50 - DNA, 78 bp, synthetic construct
  source 1..78: mol_type = other DNA
cgtggccttagctgtgctcgcgctactctctctttcgcctggaggctatccagcgtgagtctctcctaccctcccgct

SEQ ID NO: 51 - DNA, 75 bp, synthetic construct
  source 1..75: mol_type = other DNA
cgtggccttagctgtgctcgcgctactctctctttctggaggctatccagcgtgagtctctcctaccctcccgct

SEQ ID NO: 52 - DNA, 84 bp, synthetic construct
  source 1..84: mol_type = other DNA
cgtggccttagctgtgctcgcgctactctctctttctggatagcctggaggctatccagcgtgagtctctcctaccctcccgct

SEQ ID NO: 53 - DNA, 55 bp, synthetic construct
  source 1..55: mol_type = other DNA
cgtggccttagctgtgctcgcgctatccagcgtgagtctctcctaccctcccgct

SEQ ID NO: 54 - DNA, 82 bp, synthetic construct
  source 1..82: mol_type = other DNA
cgtggccttagctgtgctcgcgctactctctctttctgtggcctggaggctatccagcgtgagtctctcctaccctcccgct

SEQ ID NO: 55 - DNA, 13 bp, synthetic construct
  source 1..13: mol_type = other DNA
catgactggaaga

SEQ ID NO: 56 - DNA, 16 bp, synthetic construct
  source 1..16: mol_type = other DNA
catgacttctggaaga

SEQ ID NO: 57 - DNA, 17 bp, synthetic construct
  source 1..17: mol_type = other DNA
catgactttctggaaga

SEQ ID NO: 58 - DNA, 21 bp, synthetic construct
  source 1..21: mol_type = other DNA
catgactttatttctggaaga

SEQ ID NO: 59 - DNA, 21 bp, synthetic construct
  source 1..21: mol_type = other DNA
catgactttaattctggaaga

SEQ ID NO: 60 - DNA, 18 bp, synthetic construct
  source 1..18: mol_type = other DNA
catgacttttctggaaga

SEQ ID NO: 61 - DNA, 11 bp, synthetic construct
  source 1..11: mol_type = other DNA
catctggaaga

SEQ ID NO: 62 - DNA, 19 bp, synthetic construct
  source 1..19: mol_type = other DNA
catgactttttctggaaga

SEQ ID NO: 63 - DNA, 19 bp, synthetic construct
  source 1..19: mol_type = other DNA
catgactttatctggaaga

SEQ ID NO: 64 - DNA, 13 bp, synthetic construct
  source 1..13: mol_type = other DNA
catgactttaaga

SEQ ID NO: 65 - DNA, 10 bp, synthetic construct
  source 1..10: mol_type = other DNA
ttctggaaga

SEQ ID NO: 66 - DNA, 18 bp, synthetic construct
  source 1..18: mol_type = other DNA
catgactttactggaaga

SEQ ID NO: 67 - DNA, 21 bp, synthetic construct
  source 1..21: mol_type = other DNA
gtgggtggcaaagcgggtggt

SEQ ID NO: 68 - DNA, 19 bp, synthetic construct
  source 1..19: mol_type = other DNA
gtgggtggcagcgggtggt

SEQ ID NO: 69 - DNA, 21 bp, synthetic construct
  source 1..21: mol_type = other DNA
gtgggtggcatagcgggtggt

SEQ ID NO: 70 - DNA, 18 bp, synthetic construct
  source 1..18: mol_type = other DNA
gtgggtggagcgggtggt

SEQ ID NO: 71 - protein, 7 aa, synthetic construct
TGGYWWT

SEQ ID NO: 72 - protein, 16 aa, synthetic construct
YIFSSGNTNYNPSIKS

SEQ ID NO: 73 - protein, 9 aa, synthetic construct
AYGKLGFDY

SEQ ID NO: 74 - protein, 12 aa, synthetic construct
TLSSQHSTYTIG

SEQ ID NO: 75 - protein, 11 aa, synthetic construct
VNSDGSHSKGD

SEQ ID NO: 76 - protein, 9 aa, synthetic construct
GSSDSSGYV

SEQ ID NO: 77 - protein, 119 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVS

SEQ ID NO: 78 - protein, 111 aa, synthetic construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 79 - protein, 246 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 80 - protein, 494 aa, synthetic construct
MALPVTALLLPLALLLHAARPQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 473 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 21 aa, synthetic construct
MALPVTALLLPLALLLHAARP

SEQ ID NO: 83 - protein, 22 aa, synthetic construct
MLLLVTSLLLCELPHPAFLLIP

SEQ ID NO: 84 - protein, 42 aa, synthetic construct
KRGRKKLLYIFKQPFMRPVQTTQEEDGCSCRFPEEEEGGCEL

SEQ ID NO: 85 - protein, 40 aa, synthetic construct
SKRSRLLHSDYMNMTPRRPGPTRKHYQPYAPPRDFAAYRS

SEQ ID NO: 86 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 88 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 89 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 90 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 91 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 92 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 93 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 94 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 95 - DNA, 4322 bp, synthetic construct
  source 1..4322: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggttcagctgaaagaatctggccccggcctggttaaaccgagtcagagtttgagcttgacttgttctgtgactggattctcaataacaaccggtggatactggtggacctggatacgacaatttccgggccaaaagctcgagtggatgggctatatctttagtagcggcaacactaactataatccttcaatcaagagcaggataagtatcacccgagacaccagtaagaaccaattctttttgcagcttaacagcgttacgactgaaggtgacaccgcaaggtattactgcgcgagggcctacggaaaacttggttttgattattggggtcaagggacactggtaacggtttctagtgggggaggtggtagtggcggtggaggttccgggggtggaggatcccaacccgtccttacccagagtccttctgcgtccgctagtctcggaaatagtgttaagattacgtgcactttgagtagccaacattccacttataccatcggctggtatcagcaacacccagataaagccccgaaatatgtgatgtatgtcaattccgacggttctcattcaaaaggcgacgggatccccgacagattcagcggtagttcatctggagctcaccgctatctgtcaatatccaatattcagccggaagatgaagctgactatttttgcgggagcagtgatagttccggttatgtcttcggcagtggtacacagcttaccgtactcagagcagccgctacaacaactcctgcaccgcgacctccaacgcctgcccctaccattgcgtcacaaccacttagccttaggccagaagcgtgccgacctgcagcaggcggggccgttcatactaggggtcttgacttcgcgtgcgatatctacatatgggcacctctggcgggaacctgtggtgtgttgcttcttagtctggtaattacactctactgtaaacggggcagaaagaaactcctgtatatattcaaacaaccatttatgagaccagtacaaactactcaagaggaagatggctgtagctgccgatttccagaagaagaagaaggaggatgtgaactgcgcgtgaagttttcacgatctgcagatgccccggcatataaacaaggtcagaaccagttgtacaatgaattgaaccttggtagaagggaggagtatgacgtcctcgacaagagaagagggcgcgacccggagatgggcggtaagccaaggagaaaaaatccgcaagaggggctttataatgaactccagaaggataagatggctgaggcttacagtgagattggtatgaaaggagaaagaagacgcggaaagggacatgatgggttgtaccaagggctcagcactgctaccaaggacacttatgacgcgctccatatgcaagcccttccccctagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 96 - RNA, 97 nt, synthetic construct
  source 1..97: mol_type = other RNA
aggttccctacacggtctccgttttagagctagaaatagcaagttaaaataaggctagtccgttatcaacttgaaaaagtggcaccgagtcggtgct

SEQ ID NO: 97 - RNA, 97 nt, synthetic construct; identical to SEQ ID NO: 96
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 98 - RNA, 20 nt, synthetic construct
  source 1..20: mol_type = other RNA
aggttccctacacggtctcc

SEQ ID NO: 99 - RNA, 20 nt, synthetic construct
  source 1..20: mol_type = other RNA
  misc_feature 1..4: note = modified with 2'-O-methyl phosphorothioate
aggttccctacacggtctcc

SEQ ID NO: 100 - DNA, 23 bp, synthetic construct
  source 1..23: mol_type = other DNA
aggttccctacacggtctcctgg

SEQ ID NO: 101 - DNA, 20 bp, synthetic construct
  source 1..20: mol_type = other DNA
aggttccctacacggtctcc